SEQUENCE LISTING (15 sequences)

SEQ ID NO: 1 - protein, 21 aa, artificial sequence
  synthetic peptide
RRRRRRRRRQQPPVPPQRPMA

SEQ ID NO: 2 - protein, 5 aa, artificial sequence
  synthetic peptide
QQPPV

SEQ ID NO: 3 - protein, 11 aa, artificial sequence
  synthetic peptide
QQPPVPPQRPM

SEQ ID NO: 4 - protein, 10 aa, artificial sequence
  synthesized peptide
QQPPVPPQRP

SEQ ID NO: 5 - protein, 9 aa, artificial sequence
  synthesized peptide
QQPPVPPQR

SEQ ID NO: 6 - protein, 8 aa, artificial sequence
  synthesized peptide
QQPPVPPQ

SEQ ID NO: 7 - protein, 7 aa, artificial sequence
  synthesized peptide
QQPPVPP

SEQ ID NO: 8 - protein, 6 aa, artificial sequence
  synthesized peptide
QQPPVP

SEQ ID NO: 9 - protein, 5 aa, Artificial Sequence
  synthesized peptide
QQPPX

SEQ ID NO: 10 - DNA, 4366 bp, Homo sapiens
tgcattctttgccccaaaactctttcctttggttgtgctaagaggtgatgcccaaggtgcaccacctttcaagaactggatcatgaacaactttatcctcctggaagaacagctcatcaagaaatcccaacaaaagagaagaacttctccctcgaactttaaagtccgcttctttgtgttaaccaaagccagcctggcatactttgaagatcgtcatgggaagaagcgcacgctgaaggggtccattgagctctcccgaatcaaatgtgttgagattgtgaaaagtgacatcagcatcccatgccactataaatacccgtttcaggtggtgcatgacaactacctcctatatgtgtttgctccagatcgtgagagccggcagcgctgggtgctggcccttaaagaagaaacgaggaataataacagtttggtgcctaaatatcatcctaatttctggatggatgggaagtggaggtgctgttctcagctggagaagcttgcaacaggctgtgcccaatatgatccaaccaagaatgcttcaaagaagcctcttcctcctactcctgaagacaacaggcgaccactttgggaacctgaagaaactgtggtcattgccttatatgactaccaaaccaatgatcctcaggaactcgcactgcggcgcaacgaagagtactgcctgctggacagttctgagattcactggtggagagtccaggacaggaatgggcatgaaggatatgtaccaagcagttatctggtggaaaaatctccaaataatctggaaacctatgagtggtacaataagagtatcagccgagacaaagctgaaaaacttcttttggacacaggcaaagaaggagccttcatggtaagggattccaggactgcaggaacatacaccgtgtctgttttcaccaaggctgttgtaagtgagaacaatccctgtataaagcattatcacatcaaggaaacaaatgacaatcctaagcgatactatgtggctgaaaagtatgtgttcgattccatccctcttctcatcaactatcaccaacataatggaggaggcctggtgactcgactccggtatccagtttgttttgggaggcagaaagccccagttacagcagggctgagatacgggaaatgggtgatcgacccctcagagctcacttttgtgcaagagattggcagtgggcaatttgggttggtgcatctgggctactggctcaacaaggacaaggtggctatcaaaaccattcgggaaggggctatgtcagaagaggacttcatagaggaggctgaagtaatgatgaaactctctcatcccaaactggtgcagctgtatggggtgtgcctggagcaggcccccatctgcctggtgtttgagttcatggagcacggctgcctgtcagattatctacgcacccagcggggactttttgctgcagagaccctgctgggcatgtgtctggatgtgtgtgagggcatggcctacctggaagaggcatgtgtcatccacagagacttggctgccagaaattgtttggtgggagaaaaccaagtcatcaaggtgtctgactttgggatgacaaggttcgttctggatgatcagtacaccagttccacaggcaccaaattcccggtgaagtgggcatccccagaggttttctctttcagtcgctatagcagcaagtccgatgtgtggtcatttggtgtgctgatgtgggaagttttcagtgaaggcaaaatcccgtatgaaaaccgaagcaactcagaggtggtggaagacatcagtaccggatttcggttgtacaagccccggctggcctccacacacgtctaccagattatgaatcactgctggaaagagagaccagaagatcggccagccttctccagactgctgcgtcaactggctgaaattgcagaatcaggactttagtagagactgagtaccaggccacgggctgcagatcctgaatggaggaaggatatgtcctcattccatagagcattagaagctgccaccagcccaggaccctccagaggcagcctggcctgtggcatcagtccctgagtcaccatggaagcagcatcctgaccacagctggcagtcaagccacagctggagggtcagccaccaagctgggagctgagccagaacaggagtgatgtctctgcccttcctctagcctcttgtcacatgtggtgcacaaacctcaacctgacagctttcagacagcattcttgcacttcttagcaacagagagagacatgagtaagacccagattgctatttttattgttatttttaacatgaatctaaagtttatggttccagggactttttatttgacccaacaacacagtatcccaggatatggaggcaaggggaacaaagagcatgagtctttttccaagaaaactggtgagttaagtaagattagagtgagtgtgctctgttgctgtgatgctgtcagccacagcttcctgccgtagagaatgatagagcagctgctcacacaggaggccggatattctgagaagcagctttatgaggttttacagagtatgctgctacctctctccttgaagggagcatggcgagacccattggatggattggggtgaacagttcaggtcccatgcttggagcattgggtatctgatgtctgcaccagaacaagagaacctctgacggtggagaaccatgtggtgcaagaagagatcttaggtctcttcttttataccaagctcatcttttataccaagctgtgcaggtgactatgcctcctcttctgcacagaatgcttccaccagcatcctgagaagaaatgattacttctgaaaaacatccttttttccagcctctgggaatcagccccccctctctgcactatccgatcctcatcaacagagggcagcattgtgttggtcaatgttcccttggcgagcaattgaaacttgtttaggccctagggttgagcaattttaaggttgagactccaagtctcctaaaattctaggagagaaataaagagtctgtttttgctcaaaccatcaggatggaaacagtcaggcactgactggggtgcttccaagaggcatgagagtgcctactctggcttgagcacttctatatgcaaggtgaatatgtactgagctaggagacttccctgcaaaatctctgttcaccctgggttcacatccccatgaggtaatattattattcccattttacaaataatgtaactgaggctttaaaaagccaagacatctgcccaaagtgatggaactagaaagtctagagctggtattctagcccaaatctgtctgaccgcaatacacagattctttattcctattcgacactggcttctactgaaaatgaaacggattgcagagggaataaatacaaagatggaaagccagtaaagaagtcagtatagaaccactagcgaatagtgttgctctggcacagaccactgtggttgatggcatggccctccaacttggaataggattttccttttcctattctgtatccttaccttggtcatgttaatgactttggagttattcagttaatgaccctttaattctcacaaccaaccagtcatgttgcttgaagccatttatagacgagcttcaaagcaactttaaaagattcttctgtagaagtatgagttcttcctttaattatcattccaactttcagctgtagtcttcttgaacacttcatgaggagggacattccctgatataagagaggatggtgttgcaattggctctttctaaatcatgtgacgttttgactggcttgagattcagatgcataatttttaattataattattgtgaagtggagagcctcaagataaaactctgtcattcagaagatgattttactcagcttatccaaaattatctctgtttactttttagaattttgtacattatcttttgggatccttaattagagatgatttctggaacattcagtctagaaagaaaacattggaattgactgatctctgtggtttggtttagaaaattcccctgtgcatggtattacctttttcaagctcagattcatctaatcctcaactgtacatgtgtacattcttcacctcctggtgccctatcccgcaaaatgggcttcctgcctggtttttctcttctcacattttttaaatggtcccctgtgtttgtagagaactcccttatacagagttttggttctagttttatttcgtagattttgcattttgtaccttttgagactatgtatttatatttggatcagatgcatatttattaatgtacagtcactgctagtgttcaaaataaaaatgttacaaat

SEQ ID NO: 11 - protein, 620 aa, Homo sapiens
MNNFILLEEQLIKKSQQKRRTSPSNFKVRFFVLTKASLAYFEDRHGKKRTLKGSIELSRIKCVEIVKSDISIPCHYKYPFQVVHDNYLLYVFAPDRESRQRWVLALKEETRNNNSLVPKYHPNFWMDGKWRCCSQLEKLATGCAQYDPTKNASKKPLPPTPEDNRRPLWEPEETVVIALYDYQTNDPQELALRRNEEYCLLDSSEIHWWRVQDRNGHEGYVPSSYLVEKSPNNLETYEWYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPVCFGRQKAPVTAGLRYGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 12 - protein, 7 aa, artificial sequence
  synthesized peptide
XQQPPVX

SEQ ID NO: 13 - protein, 7 aa, artificial
  synthetic peptide
XQQPPXX

SEQ ID NO: 14 - protein, 7 aa, artificial sequence
  synthetic sequence
XQQPPXX

SEQ ID NO: 15 - protein, 7 aa, artificial sequence
  synthetic sequence
XXXQPXX

(19) United States
(12) Patent Application Publication
(10) Pub. No.: US 2010/0287636 A1
(43) Pub. Date: Nov. 11, 2010

(54) COMPOSITIONS AND METHODS FOR INHIBITING INDUCIBLE T CELL KINASE (ITK) AND TREATING ASTHMA AND BRONCHIAL INFLAMMATIONS

(75) Inventors: Constantine Tsoukas, San Diego, CA (US); John Lambris, Bryn Mawer, PA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: San Diego State University Foundation, dba San Diego State University Research Foundation, San Diego, CA (US)

(21) Appl. No.: 12/600,641

(22) PCT Filed: May 15, 2008
(86) PCT No.: PCT/US08/63633

§ 371 Date: Jul. 23, 2010

Related U.S. Application Data
(60) Provisional application No. 60/939,019, filed on May 18, 2007.

(51) Int. Cl. A01K 67/027; C07K 7/06; C07K 7/08; C07K 14/00; A61K 38/10; A61K 38/16; A61P 11/06; C07K 16/00; A61P 11/08; A61K 38/08; A61K 39/395; A61K 9/127; C07H 21/04; C12N 15/63; C12N 5/10; C12N 7/01; A61K 31/7088; A61M 15/00; A61M 11/00

(52) U.S. Cl. 800/18; 530/329; 530/328; 530/327; 530/326; 530/325; 530/324; 530/387.3; 530/350; 514/1.1; 424/133.1; 424/450; 536/23.1; 435/320.1; 435/325; 435/235.1; 800/14; 514/44 A; 128/203.12; 128/200.14; 128/203.16

(57) ABSTRACT

The invention provides methods and compositions for the treatment of asthma and bronchial inflammation, e.g., as induced by an allergen or toxin. In one aspect, the invention provides inhibitors of “Inducible T Cell Kinase” (ITK) polypeptides and methods of making and using them, e.g., as agents and pharmaceutical compositions to treat asthma. In one aspect, the invention is directed to ITK protein expression and/or activity inhibitors. In one aspect, these ITK protein expression and/or activity inhibitors are used with targeting agents. In one aspect, the ITK protein inhibitors of the invention are used to treat asthma. In one aspect, the invention is directed to ITK protein inhibitors as chimeric proteins comprising fragments or altered or truncated forms of ITK protein, or equivalent. In other aspects ITK protein is joined or fused to another moiety (e.g., a targeting domain) or to an antibiotic. The invention also provides pharmaceutical compositions comprising the ITK protein inhibitors of the invention, and methods of making and using them, including methods for ameliorating or preventing asthma. The invention also provides compositions for transfecting cells with nucleic acids acting as ITK protein inhibitors and/or the chimeric ITK protein inhibitors polypeptides of the invention.

### TECHNICAL FIELD

This invention relates to medicine, molecular and cellular biology and biochemistry. In one aspect, the invention provides methods and compositions for the treatment of asthma and bronchial inflammation, e.g., as induced by an allergen or toxin. In one aspect, the invention provides suppressors and/of inhibitors of “Inducible T Cell Kinase” (ITK) polypeptides and methods of making and using them, e.g., as agents and pharmaceutical compositions to treat asthma. In one aspect, the invention is directed to ITK protein expression and/or activity suppressors or inhibitors. In one aspect, these ITK protein expression and/or activity inhibitors or suppressors are used with targeting agents. In one aspect, the ITK protein inhibitors or suppressors of the invention are used to treat asthma. In one aspect, the invention is directed to ITK protein inhibitors or suppressors as chimeric proteins comprising fragments or altered or truncated forms of ITK protein, or equivalent. In other aspects ITK protein is joined or fused to another moiety (e.g., a targeting domain) or to an antibiotic. The invention also provides pharmaceutical compositions comprising the ITK protein inhibitors or suppressors of the invention, and methods of making and using them, including methods for ameliorating, treating, suppressing or preventing asthma or asthmatic attacks. The invention also provides compositions for transfecting cells with nucleic acids acting as ITK protein inhibitors or suppressors of the invention and/or the chimeric ITK protein inhibitors or suppressors polypeptides of the invention.

### BACKGROUND

Bronchial asthma is considered a serious public health problem worldwide with a significant economic and social impact. In North America alone about 10% of the population suffers from Bronchial Asthma. The financial burden of Bronchial Asthma has been estimated to be $300-$1,300 per patient per year. In the United States the overall costs for treating Bronchial Asthma are estimated to be $10 billion annually, with another $3 billion in indirect costs associated with loss of productivity. Therefore, it is evident that pharmacological control of asthma is a quite significant issue.

Bronchial Asthma is an immunologically based disease that involves an inflammatory response in the airway. In the Western Hemisphere one of the common causes of asthma is in response to environmental allergens. Among the various cell types involved in the pathogenesis of Bronchial Asthma are CD4-positive T lymphocytes, Dendritic Cells, and Eosinophils. In a simplified model of the mechanism of Bronchial Asthma, dendritic cells in the bronchial mucosa detect an allergen that is endocytosed and carried to the local lymph nodes where it is presented to specific CD4 T cells. The CD4 T cells become activated and localize to the bronchial tissue where they secrete type 2 cytokines (e.g. IL4 and IL5) thus causing the recruitment and activation of other inflammatory cells, most notably eosinophils. These cells secrete pharmacological mediators (e.g. Leukotrienes, Prostaglandins), which are the direct mediators of the inflammatory response and symptomatology of the disease.

Current therapy for Bronchial Asthma utilizes non-specific inhibitors of inflammation such as corticosteroids, which have significant side effects. Newer therapies at various stages of development include antagonists of receptors that are involved in the adhesion of inflammatory cells to the bronchial endothelium as well as inhibitors of inflammatory molecule production such as Leukotrienes and Prostaglandins. It remains to be seen how effective these strategies will be in the treatment of asthma.

It has been almost eighteen years since the original observation that the HIV Tat transactivator protein was shown to be capable to translocate across the plasma membrane. This property prompted investigators to use Tat-fusion proteins as vehicles for the delivery of specific proteins or peptides across cell membranes. It was later discovered that the translocation property of Tat was due to an arginine-rich core at the N-terminus proximal end of the protein that was sufficient for the membrane translocation. This further prompted researchers to test whether arginine homo-oligomers could translocate across the plasma membrane. It was discovered that oligomers of seven to nine arginine residues entered cells much more efficiently than Tat itself. Following these observations several groups constructed conjugates of specific peptides with poly-arginine and tested their ability to translocate into cells. Notable among these studies is the conjugation of polyarginine to cyclosporin A and its successful entry into dermal T cells and inhibition of cutaneous inflammation. Another study inhibited T cell activation both in vitro and in vivo by using a conjugate of a polyarginine and a NFAT inhibitor peptide. This polyarginine conjugate was able to enter T cells and inhibit their antigen-mediated activation in vitro, and upon intraperitoneal administration in mice the conjugate peptide was able to inhibit T cell activation in vivo.

The idea of adding arginine to peptides to make them capable of penetrating cells dates back to the observation that a protein from HIV, the virus that causes AIDS, is able to penetrate cells. It was discovered that this property was due to the presence of arginines in the HIV protein. Further development of this technology indicated that one could add several arginines to any peptide and make it capable of penetrating cells. Investigators have successfully used the arginine delivery approach to inhibit the activation of immune cells both in laboratory situations and in live animals.

### SUMMARY

The invention provides compositions and methods for the treatment, amelioration, prevention or suppression of asthma, asthmatic attacks, respiratory allergic reactions or other pathologic responses, e.g., as induced by an allergen, irritant, poison or toxin. The invention provides methods and compositions for the treatment, amelioration, prevention or suppression of bronchial inflammation, e.g., as induced by an allergen, irritant, poison or toxin.

In one aspect, the invention provides inhibitors of “Inducible T Cell Kinase” (ITK) polypeptides and methods of making and using them, e.g., as agents and pharmaceutical compositions to treat asthma, asthmatic attacks, respiratory allergic reactions or other pathologic responses. In one aspect, the invention is directed to ITK protein expression and/or activity inhibitors. In one aspect, these ITK protein expression and/or activity inhibitors are used with targeting agents. In one aspect, the ITK protein inhibitors of the invention are used to treat asthma, asthmatic attacks, respiratory allergic reactions or other pathologic responses. In one aspect, the invention is directed to ITK protein inhibitors as chimeric proteins comprising fragments or altered or truncated forms of ITK protein, or equivalent. In other aspects ITK protein is joined or fused to another moiety (e.g., a targeting domain) or to an antibiotic. The invention also provides pharmaceutical compositions comprising the ITK protein inhibitors of the invention, and methods of making and using them, including methods for ameliorating or preventing asthma, asthmatic attacks, respiratory allergic reactions or other pathologic responses. The invention also provides compositions for transfecting cells with nucleic acids acting as ITK protein inhibitors and/or the chimeric ITK protein inhibitors polypeptides of the invention.

The invention provides compositions for transfecting nucleic acids into a cell comprising an ITK protein inhibitor of the invention. In one aspect, the ITK protein inhibitor of the invention comprises a nucleic acid comprising naked DNA or RNA, and optionally the naked DNA or RNA, or RNAi such as siRNA or miRNA, is operably linked to a promoter. In one aspect, the nucleic acid comprises plasmid DNA, a recombinant virus or phage, an expression cassette or a vector such as an expression vector. In one aspect, the cell is a bacterial cell or a mammalian cell, wherein optionally the mammalian cell is a human cell.

The invention provides methods for transfecting a cell with nucleic acid of the invention comprising the following steps: (a) providing a nucleic acid-comprising composition of the invention (for transfecting nucleic acids); (b) contacting the cell with the composition of step (a) under conditions wherein the composition is internalized into the cell. In one aspect, the transfecting is an in vivo transfection or an in vitro transfection.

The invention provides methods for preventing, inhibiting, suppressing or ameliorating inflammation of bronchial tubes by inhibiting the activation of T-helper cells responsible for the inflammation signaling pathway, the method comprising, a peptide conjugate that is able to efficiently gain entry into T-helper cell and inhibit the activation of ITK and SLP-76, leading to inhibition of T-helper cell responsible for activating the signaling pathway responsible for bronchial inflammation. In one aspect, the inflammation of bronchial asthma that is prevented, inhibited, suppressed or ameliorated is caused by an immune response to the inhalation of allergens, e.g., where inhaled allergens bind to and activate T-helper cells. In one aspect, the T-helper cell whose activation is inhibited, suppressed or prevented is a CD4 positive T cell, or any leukocyte that when activated moves to the lungs and signals other immune cells. In one aspect, the prevention, suppression or inhibition of the activation of CD4 positive T cells leads to prevention, suppression or inhibition of the activation of other inflammatory cells and secretion of prostaglandins, type-2 cytokines and/or leukotrienes.

In one aspect, an exemplary peptide conjugate of the invention is a Poly-ArgSLP76. In one aspect, the polypeptides and peptides of the invention have increased intracellular penetration and delivery because of several arginines (at least two, or two to twenty) added to the peptide SLP76. In one aspect, the poly-arginine complex consists of 7 to 9 oligomers of arginine.

In one aspect, the polypeptides and peptides of the invention comprise a peptide conjugate consisting of the subsequence of SLP76 that binds to ITK. In one aspect, the polypeptides and peptides of the invention are able to efficiently penetrate and enter CD4 positive T cells because the poly-arginine complex adds a positive charge.

In one aspect, the polypeptides and peptides of the invention inhibit the binding of ITK to SLP-76 inside a T cell, thus causing inhibition of the activation of ITK, and thus causing inhibition of activation of the T cell. In one aspect, the methods and compositions of the invention, by preventing the binding of SLP76 to ITK, inhibit the activation of the signaling pathway responsible for inflammation of bronchial tubes.

The invention provides isolated, synthetic or recombinant polypeptides or peptides comprising or consisting of

(a) an amino acid sequence comprising or consisting of the formula:

R₁-QQPPV (SEQ ID NO: 2)-R₂,
R₁-GlnGlnProProVal (SEQ ID NO: 2)-R₂,
or
R₁-GlnGlnProProR₃ (SEQ ID NO: 9)-R₂,
or
R₁-GlnGlnProProR₄ (SEQ ID NO: 9)-R₂,
or
R₁-R₅R₆GlnProProR₄ (SEQ ID NO: 9)-R₂,

wherein in R₁ and R₂, R is independently an arginine amino acid residue, an arginine peptidomimetic residue, a ketopiperazine, an amidinophenylalanine residue, a guanidino group-containing basic amino acid or an arginine amino acid equivalent; and 1 and 2 of R₁ and R₂ is an integer between 1 and 50, or 1 and 2 of R₁ and R₂ are independently selected from 1, 2, 3, 4, 5, 6, 7, 8, 9, 10, 11, 12, 13, 14, 15, 16, 17, 18, 19 or 20 or more arginine amino acid residues, ketopiperazine residues, amidinophenylalanine residues, arginine peptidomimetics residues or arginine amino acid equivalents,

and R₃ is a hydrophobic amino acid residue, or R₄ is valine (val, or V), leucine (Leu, or L), isoleucine (Ile, or I), or alanine (ala, or A), or equivalent;

and R₄ is a nonpolar amino acid residue, or R₄ is glycine (Gly, or G), alanine (ala, or A), valine (val, or V), leucine (Leu, or L), isoleucine (Ile, or I), methionine (met, or M), phenylalanine (Phe, or F), tryptophan (trp, or W) or proline (pro, or P), or equivalent;

and R₅ and R₆ are a polar amino acid residue, or R₅ and R₆ are independently serine (ser, or S), threonine (thr, or T), cysteine (cys, or C), tyrosine (tyr, or Y), asparagine (asp, or N), or glutamine (Gln, or Q);

| | |
|---|---|
| (b) amino acid sequence | |
| RRRRRRRRRQQPPVPPQRPMA, | (SEQ ID NO: 1) |
| QQPPV, | (SEQ ID NO: 2) |
| QQPPVPPQRPM, | (SEQ ID NO: 3) |
| QQPPVPPQRP; | (SEQ ID NO: 4) |
| QQPPVPPQR, | (SEQ ID NO: 5) |
| QQPPVPPQ, | (SEQ ID NO: 6) |
| QQPPVPP, | (SEQ ID NO: 7) |
| or | |
| QQPPVP; | (SEQ ID NO: 8) |

(c) the polypeptide or peptide of (b) further comprising a poly-arginine amino acid residue moiety, or equivalent;

(d) the polypeptide or peptide of (c), wherein the poly-arginine moiety amino acid residues are located amino terminal, carboxy terminal, or amino terminal and carboxy terminal to the polypeptide or peptide;

(d) a polypeptide or peptide conjugate comprising a non-functional subsequence of SEQ ID NO:1, SEQ ID NO:3, SEQ ID NO:4, SEQ ID NO:5, SEQ ID NO:6, SEQ ID NO:7 or SEQ ID NO:8, and comprising the motif QQPPV (SEQ ID NO:2) and a poly-arginine moiety, or equivalent;

(c) a peptidomimetic of the polypeptide or peptide of any of (a) to (d).

The invention provides chimeric proteins comprising

(A) (a) a first domain comprising the isolated, synthetic or recombinant polypeptide or peptide of the invention, and at least a second domain or moiety; (b) the chimeric protein of (a), wherein the chimeric protein comprises a recombinant fusion protein; or (c) the chimeric protein of (a) or (b), wherein the second domain or moiety comprises a targeting agent.

(B) the chimeric protein of (A), wherein the targeting agent comprises an antibody Fc domain or an antibody that binds to an Fc receptor, or a chimeric protein comprising two or more antibody Fc domains;

(C) the chimeric protein of (A) or (B), wherein the at least second domain or moiety comprises an Fc domain, a protein C, an antibacterial or bacteriostatic peptide or protein, an antibiotic, a cytokine, an immunoregulatory agent, an anti-inflammatory agent, a complement activating agent, a carbohydrate-binding domain or a combination thereof;

(D) the chimeric protein of any of (A) to (C), wherein the chimeric protein comprises a recombinant, peptidomimetic or synthetic protein; or

(E) the chimeric protein of any of (A) to (D), wherein the first domain is joined to the second domain or moiety by a chemical linking agent.

The invention provides compositions comprising (a) a first composition comprising the isolated, synthetic or recombinant polypeptide or peptide of the invention, or the chimeric protein of the invention; and a second composition; or (b) the composition of (a), wherein the second composition comprises a liquid, a lipid or a powder.

The invention provides liposomes comprising (a) the isolated, synthetic or recombinant polypeptide or peptide of the invention, or the chimeric protein of the invention; or (b) the liposome of (a), wherein the liposome is formulated with a pharmaceutically acceptable excipient.

The invention provides pharmaceutical compositions comprising: the isolated, synthetic or recombinant polypeptide or peptide of the invention, or the chimeric protein of the invention, the composition of the invention, or the liposome of the invention; and, a pharmaceutically acceptable excipient.

The invention provides inhalants or spray formulations comprising: the isolated, synthetic or recombinant polypeptide or peptide of the invention, or the chimeric protein of the invention, or the composition of the invention, or the liposome of the invention, or the pharmaceutical composition of the invention; and, a pharmaceutically acceptable excipient.

The invention provides formulations comprising: the isolated, synthetic or recombinant polypeptide or peptide of the invention, or the chimeric protein of the invention, or the composition of the invention, or the liposome of the invention, or the pharmaceutical composition of the invention; and, a pharmaceutically acceptable excipient; wherein the formulation can be a parenteral or enteral formulation. For example, the invention provides enteral formulations comprising: the isolated, synthetic or recombinant polypeptide or peptide of the invention, or the chimeric protein of the invention, or the composition of the invention, or the liposome of the invention, or the pharmaceutical composition of the invention; and, a pharmaceutically acceptable excipient.

The invention provides methods for treating, ameliorating or preventing a bronchial inflammation and/or an asthma or asthmatic attack, or any respiratory allergic reaction or other pathologic respiratory response, in an individual in need thereof, comprising:

(A) (a) providing the isolated, synthetic or recombinant polypeptide or peptide of the invention, the chimeric protein of the invention, the composition of the invention, the liposome of the invention, the pharmaceutical composition of the invention, the inhalant or spray formulation of the invention, the parenteral formulation of the invention, or the enteral formulation of the invention; and (b) administering an effective amount of (a) to the individual, thereby preventing, ameliorating or treating the bronchial inflammation and/or an asthma or asthmatic attack, or any respiratory allergic reaction or other pathologic respiratory response; or,

(B) the method of (A), wherein the asthma is a human bronchial asthma, and/or the bronchial inflammation and/or an asthma or asthmatic attack, or any respiratory allergic reaction or other pathologic respiratory response, is caused by a toxin, poison or poison gas, and/or allergen.

The invention provides methods for ameliorating, preventing or suppressing inflammation of bronchial tubes by ameliorating, preventing or suppressing the activation of T-helper cells in an individual, comprising:

(A) (a) providing the isolated, synthetic or recombinant polypeptide or peptide of the invention, the chimeric protein of the invention, the composition of the invention, the liposome of the invention, the pharmaceutical composition of the invention, the inhalant or spray formulation of the invention, the parenteral formulation of the invention, or the enteral formulation of the invention; and (b) administering an effective amount of the composition of (a) to the individual; or (B) the method of (A), wherein the individual is a human.

The invention provides methods for ameliorating, preventing, suppressing or treating an immune response in an individual in response to inhalation of an allergen, irritant or toxin by the individual, comprising:

(A) (a) providing the isolated, synthetic or recombinant polypeptide or peptide of the invention, the chimeric protein of the invention, the composition of the invention, the liposome of the invention, the pharmaceutical composition of the invention, the inhalant or spray formulation of the invention, the parenteral formulation of the invention, or the enteral formulation of the invention; and (b) administering an effective amount of the composition of (a) to the individual to ameliorate, prevent, suppress or treat the immune response (that was in response to inhalation of an allergen, irritant or toxin (including poison or poison gas) by the individual); or

(B) the method of (A), wherein the individual is a human.

The invention provides methods for ameliorating, suppressing or preventing activation of a CD4⁺ T cell, comprising:

(A) (a) providing the isolated, synthetic or recombinant polypeptide or peptide of the invention, the chimeric protein of the invention, the composition of the invention, the liposome of the invention, the pharmaceutical composition of the invention, the inhalant or spray formulation of the invention, the parenteral formulation of the invention, or the enteral formulation of the invention; and (b) contacting an effective amount of the composition of (a) to the CD4⁺ T cell; or

(B) the method of (A), wherein the CD4⁺ T cell is isolated or in an individual with asthma, bronchial asthma, a bronchial inflammation, or a bronchial inflammation caused by a toxin, poison or poison gas, and/or allergen.

The invention provides methods for suppressing or preventing secretion from an inflammatory cell a prostaglandin, a type-2 cytokines and/or a leukotriene, comprising:

(A) (a) providing the isolated, synthetic or recombinant polypeptide or peptide of the invention, the chimeric protein of the invention, the composition of the invention, the liposome of the invention, the pharmaceutical composition of the invention, the inhalant or spray formulation of the invention, the parenteral formulation of the invention, or the enteral formulation of the invention; and (b) contacting an effective amount of the composition of (a) to the inflammatory cell; or

(B) the method of (A), wherein the inflammatory cell is isolated or in an individual with asthma, bronchial asthma, a bronchial inflammation, or a bronchial inflammation caused by a toxin, poison or poison gas, and/or allergen.

The invention provides isolated, synthetic or recombinant nucleic acids comprising or consisting of:

(a) a nucleic acid sequence encoding the polypeptide or peptide of the invention;

(b) the nucleic acid sequence of (a), and further comprising or consisting of nucleic acid sequence encoding a polypeptide antigen, label or tag;

(c) the nucleic acid sequence of (b), wherein the polypeptide antigen, label or tag comprises or consists of a fluorescent or a detectable protein, or an enzyme, or an enzyme that generates a detectable agent or moiety.

The invention provides vectors, cloning or expression vectors, expression cassettes, plasmids, phages, or recombinant viruses comprising the isolated or recombinant nucleic acid of the invention.

The invention provides host cells comprising (a) the vector, cloning or expression vector, expression cassette, plasmid, phage, or recombinant virus of the invention, or a recombinant nucleic acid encoding the polypeptide of the invention; or a nucleic acid of the invention; or (b) the host cell of (a), wherein the cell is a bacterial cell, a mammalian cell, a fungal cell, an insect cell, a yeast cell or a plant cell.

The invention provides non-human transgenic animals comprising (a) the vector, cloning or expression vector, expression cassette, plasmid, phage, or recombinant virus of the invention, or a recombinant nucleic acid encoding the polypeptide of the invention; or a nucleic acid of the invention; or (b) the non-human transgenic animal of (a), wherein the animal is a mouse or a rat.

The invention provides methods for transfecting a cell with a nucleic acid comprising: (a) providing a nucleic acid encoding a polypeptide of the invention, or a nucleic acid of the invention; and, (b) contacting the cell with the nucleic acid of (a) under conditions wherein the nucleic acid is internalized into the cell.

The invention provides pharmaceutical compositions comprising (a) a human ITK antisense inhibitory nucleic acid; and, a pharmaceutically acceptable excipient, wherein the antisense inhibitory nucleic acid is inhibitory to the expression of SEQ ID NO:10, or (b) the human ITK antisense inhibitory nucleic acid of (a), comprising or consisting of an iRNA, an miRNA, an siRNA, an antisense nucleic acid and/or a ribozyme.

The invention provides inhalants or spray formulations comprising the pharmaceutical composition of the invention; and, a pharmaceutically acceptable excipient. The invention provides parenteral formulations comprising the pharmaceutical composition of the invention; and, a pharmaceutically acceptable excipient. The invention provides enteral formulations comprising the pharmaceutical composition of the invention; and, a pharmaceutically acceptable excipient.

The invention provides methods for treating, ameliorating or preventing a bronchial inflammation and/or an asthma, or an asthmatic incident, in an individual in need thereof, comprising:

(A) (a) providing the pharmaceutical composition of the invention; and (b) administering an effective amount of (a) to the individual, thereby treating the asthma; or,

(B) the method of (A), wherein the asthma is a human bronchial asthma, and/or the bronchial inflammation is caused by a toxin, poison or poison gas, and/or allergen.

The invention provides methods for suppressing inflammation of bronchial tubes by inhibiting the activation of T-helper cells in an individual, comprising: (A) (a) providing the pharmaceutical composition of the invention; and (b) administering an effective amount of the composition of (a) to the individual; or (B) the method of (A), wherein the individual is a human.

The invention provides methods for suppressing or preventing an immune response in an individual in response to inhalation of an allergen by the individual, comprising: (A) (a) providing the pharmaceutical composition of the invention; and (b) administering an effective amount of the composition of (a) to the individual; or (B) the method of (A), wherein the individual is a human.

The invention provides methods for suppressing or preventing activation of a CD4⁺ T cell, comprising: (A) (a) providing the pharmaceutical composition of the invention; and (b) contacting an effective amount of the composition of (a) to the CD4⁺ T cell; or (B) the method of (A), wherein the CD4⁺ T cell is isolated or in an individual with asthma, bronchial asthma, a bronchial inflammation, or a bronchial inflammation caused by a toxin, poison or poison gas, and/or allergen.

The invention provides methods for suppressing or preventing secretion from an inflammatory cell a prostaglandin, a type-2 cytokines and/or a leukotriene, comprising: (A) (a) providing the pharmaceutical composition of the invention; and (b) contacting an effective amount of the composition of (a) to the inflammatory cell; or (B) the method of (A), wherein the inflammatory cell is isolated or in an individual with asthma, bronchial asthma, a bronchial inflammation, or a bronchial inflammation caused by a toxin, poison or poison gas, and/or allergen.

The invention provides methods an inhaler, nebulizer or atomizer comprising pharmaceutical composition of the invention.

The invention provides uses of an isolated, synthetic or recombinant polypeptide or peptide of the invention, the chimeric protein of the invention, the composition of the invention, the liposome of the invention, or the inhalant or spray formulation of the invention to make a pharmaceutical composition. In one aspect, the pharmaceutical composition is made to treat, prevent or ameliorate asthma, a bronchial inflammation, or a bronchial inflammation induced by an allergen, a toxin, a poison, a gas or an irritant.

The invention provides uses an ITK-inhibitory nucleic acid or peptide or polypeptide of the invention to make a pharmaceutical composition. In one aspect, the pharmaceutical composition is made to treat, prevent or ameliorate asthma, a bronchial inflammation, or a bronchial inflammation induced by an allergen, a toxin, a poison, a gas or an irritant.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 illustrates a picture of immune complexes resolved by gel electrophoresis; these data demonstrating that upon T cell stimulation, immunoprecipitation of SLP76 also co-immunoprecipitates ITK, as discussed in detail in Example 1, below.

FIG. 2 illustrates a picture of immune complexes resolved by gel electrophoresis; Jurkat T cells were activated through their TCR for various periods of time as indicated, lysed, and then analyzed by immunoprecipitation; ITK was immunoprecipitated with specific anti-ITK antibodies and after resolution by gel electrophoresis, it was probed with anti-phosphotyrosine antibodies (IB: anti-pY); as discussed in detail in Example 1, below.

FIG. 3 illustrates a picture of flow cytometry (FIG. 3A, top panel) or confocal microscopy (bottom panels) data from Jurkat cells; FIG. 3A illustrates a picture of flow cytometry incubated with 1 μM, or 3 μM of a fluoresceinated peptide of this invention, called R9-QQP, or a buffer control (0 μM peptide), the amount of fluorescence intensity is graphically illustrated; for the confocal microscopy (FIG. 3B, bottom panels), Panel A (of FIG. 3B) displays the DIC image of the field; panel B (of FIG. 3B) is an overlay of the fluorescence and differential interference contrast (DIC) images; and panel C (of FIG. 3B) is a zoom image; as discussed in detail in Example 1, below.

FIG. 4 illustrates a picture of immune complexes resolved by gel electrophoresis, where Jurkat cells were stimulated as described for FIG. 1, in the presence or absence of various concentrations of the R9-QQP peptide of the invention; following incubation cells were lysed and SLP76 was pulled down with anti-SLP76 specific antibodies (IP: anti-SLP 76), and the protein complexes were resolved by gel electrophoresis and then probed with anti-ITK antibodies (IB: anti-ITK) or with anti-SLP 76 antibodies (IB: anti-SLP76); as discussed in detail in Example 1, below.

FIG. 5 is a graphic illustration of data depicting the average of three experiments as described for FIG. 4, and in this figure displayed as Association Index vs. peptide concentration; where the association index is defined as in FIG. 4 above=the ratio of the ITK signal over the total SLP 76 signal; as discussed in detail in Example 1, below.

FIG. 6 is a graphic illustration of data demonstrating that ITK phosphorylation is inhibited by the exemplary peptide of the invention R9-QQP in a dose-dependent fashion; as discussed in detail in Example 1, below.

FIG. 7 is a graphic illustration of data demonstrating that the in vivo delivery of a R9-QQP peptide of this invention disrupted the stimulation-induced association between ITK and SLP 76; and that the inhibition of ITK and SLP 76 interaction by this peptide of this invention is highly specific for ITK; as discussed in detail in Example 1, below.

FIG. 8 is a graphic illustration of data demonstrating that ITK phosphorylation is inhibited in a dose-dependent manner by the exemplary R9-QQP peptide of the invention; as discussed in detail in Example 1, below.

FIG. 9 is a graphic illustration of data demonstrating that the exemplary R9-QQP peptide of this invention specifically inhibits type 2 cytokines that are known to be regulated by ITK; as discussed in detail in Example 1, below.

FIG. 10 illustrates an exemplary synthesis of an arginine peptidomimetic for use in this invention.

Like reference symbols in the various drawings indicate like elements.

### DETAILED DESCRIPTION

The invention provides compositions and methods for the treatment of bronchial asthma, asthmatic attacks, or any respiratory allergic reaction or other pathologic respiratory response. In one aspect, the invention provides inhibitors or suppressors of “Inducible T Cell Kinase” (ITK) polypeptides and methods of making and using them, e.g., as agents and pharmaceutical compositions to treat asthma. The invention provides compositions and methods to mediate a major cellular component of the allergic response in bronchial asthma—the CD4-T lymphocyte. The inventors' studied the activation of these cells; focusing on an intracellular protein tyrosine kinase in T cells, known as the Inducible T cell Kinase (abbreviated ITK), a multidomain protein composed of SH2, SH3 and SH1 (kinase) domains (see e.g., reference (11), below). In addition, ITK encompasses a PH domain that allows its interaction of with the cell membrane.

The invention provides ITK inhibitory or suppressing compositions and methods to treat asthma, asthmatic attacks, or any respiratory allergic reaction or other pathologic respiratory response. While the invention is not limited by any particular mechanism of action, in one aspect, the ITK inhibitory compositions and methods of the invention are effective because it has been demonstrated that ITK plays a critical role in the activation of CD4 T cells and in the secretion of type 2 cytokines, such as those involved in the pathogenesis of Bronchial Asthma. Additionally, the efficacy of the ITK inhibitory compositions and methods of this invention also is validated by a strain of mice deficient in the expression of the gene that codes for ITK (ITK-KO mice); these mice are an art-accepted animal model that have been used to show protection against Bronchial Asthma as an experimental animal model of induced asthma. Thus, in contrast to their normal (wild type) counterparts, ITK-KO mice do not develop the inflammatory responses and profound eosinophilia in response to an allergic challenge. The invention provides ITK inhibitory compositions and methods effective for ameliorating (including treating) and preventing asthma, or treating or preventing asthmatic attacks or related bronchial incidents, as ITK is a critical component for the transcriptional activation of type 2 cytokines necessary for the induction of an asthmatic response.

Accordingly, in alternative embodiments, compositions and methods of the invention are used to prevent, ameliorate or treat asthma, asthmatic attacks, or any respiratory allergic reactions or other pathologic respiratory responses triggered by such things as exposure to an environmental stimulant, e.g., an irritant, poison, toxin, or allergen, including cold air, warm or hot air, perfume, moist air, or exercise or exertion, or emotional stress. In individuals, particularly children, compositions and methods of the invention are used to prevent, ameliorate or treat asthma, asthmatic attacks, or any respiratory allergic reactions or other pathologic respiratory responses triggered by viral illnesses such as those that caused by flu or the common cold. In alternative embodiments, compositions and methods of the invention are used to prevent, ameliorate or treat airway narrowing, wheezing, shortness of breath, chest tightness, and coughing caused by, e.g., exposure to an environmental stimulant, e.g., an irritant, poison, toxin, or allergen, including cold air, warm or hot air, perfume, moist air, or exercise or exertion, or emotional stress, or asthma, asthmatic attacks, or any respiratory allergic reactions or other pathologic respiratory conditions.

In alternative embodiments, compositions and methods of the invention are administered in conjunction with other treatments or drugs for asthma, asthmatic attacks, or any respiratory allergic reactions or other pathologic respiratory conditions, such as steroids, long-acting bronchodilators (LABD), such as long-acting beta₂-adrenoceptor agonists, or short-acting selective beta₂-adrenoceptor agonists, such as salmeterol, formoterol, bambuterol or sustained-release oral albuterol, or the combination of budesonide and formoterol.

In view of the observations in ITK-deficient mice, inhibiting the activation of ITK in vivo (mimicking the ITK null situation of the ITK-KO animals) will ameliorate, treat, suppress or prevent bronchial or respiratory allergic, irritant or toxin challenges in an individual, e.g., a human. Thus, the invention provides suppressors and/or inhibitors of ITK activation. The effectiveness of these suppressors and/o inhibitors can be validated in both cell line-based systems and in vivo by injecting them into animals. Compositions of the invention can be delivered directly or indirectly to a site of inflammation, which in the case of Bronchial Asthma is the bronchial mucosa. The invention provides “treatment” for asthma, including the partial or complete amelioration of at least one symptom of, partially or completely treating or curing and/or preventing the development of a related disease or a condition, for example, asthma or an asthmatic attack.

Thus, the invention provides pharmaceutical compositions and methods for the partial or complete amelioration of at least one symptom of, partially or completely treating or curing and/or preventing the development of bronchial asthma, an immunologically based disease that involves an inflammatory response in the airway. The compositions and methods of the invention can be used prophylactically if one is aware of possible exposure to a common cause of asthma, e.g., an environmental allergen.

While the invention is not limited by any particular mechanism of action, it provides pharmaceutical compositions and methods for the partial or complete amelioration of the pathogenesis of asthma in response to allergens as mediated by a specific type of white blood cell known as the CD4-T cell. The compositions and methods of the invention can be used to stop or mediate allergens from activating CD4-T cells that move to the lungs and signal to other inflammatory cells to infiltrate the lungs. The compositions and methods of the invention can be used to stop or mediate cells from secreting pharmacological mediators, e.g., cytokines, leukotrienes and prostaglandins that create the symptoms of Bronchial Asthma. This invention's methods can also be practiced with current therapies for bronchial asthma, e.g., those that utilize non-specific inhibitors of inflammation such as corticosteroids, which have significant side effects, or antagonists of receptors that are involved in the movement of inflammatory cells to the bronchial endothelium, and/or with inhibitors of inflammatory molecule production, such as leukotrienes and prostaglandins (3).

This invention found compositions to control the activation of CD4-T cells and the CD4-T cell component known as ITK, known to be important for the activation of CD4-T cells and for the production of the mediators that cause inflammation, such as the one seen in bronchial asthma. To accomplish the inhibition of ITK, this invention capitalized on the observation that for ITK to become active it must bind and interact with another cellular component known as SLP76. Thus, this invention found that if the binding and interaction between ITK and SLP76 is blocked, activation of ITK is inhibited, and thus the ability of CD4-T cells to make inflammatory mediators such as those causing bronchial asthma is inhibited. The inventors synthesized a portion of SLP76 as a synthetic peptide (based on the exact site on SLP76 onto which ITK binds) for its use as a potential inhibitor of the interaction between the two molecules ITK and SLP76.

Because the interaction of ITK and SLP76 occurs within CD4-T cells, the invention provides methods for placing this inhibitory synthetic peptide inside the appropriate cells. In one aspect, the invention provides methods for penetrating the cell membrane with this inhibitory synthetic peptide to get it inside the appropriate cells. In one aspect, a positive charge is added to the ITK inhibitory peptides of the invention such that they can easily penetrate a desired cell membrane to get inside cells. In one aspect, this positive charge is created by the addition of several copies of the amino acid arginine.

The inventors modified an ITK inhibitory peptide, wherein the peptide represents the SLP76 site onto which ITK binds, by adding nine arginines to it. Upon testing the inventors found that the modified peptide enters T cells very efficiently. The inventors further found that upon entry the peptide could inhibit the interaction between ITK and its partner SLP76. Thus, the peptide of this invention is a potent inhibitor of the action of ITK. The effectiveness of this peptide can be demonstrated in both laboratory conditions and in animals where the activity of ITK and CD4-T cells is measured.

In alternative embodiments, the invention provides 1) polyArg-SLP76 peptide conjugate, 2) efficient penetration and entry of the peptide inside living cells, 3) efficient inhibition of the interaction between ITK and SLP76 inside living cells.

The invention provides specific inhibitors of ITK action, where ITK is a critical cellular component that regulates the production of inflammatory mediators, particularly those responsible for the pathogenesis of bronchial asthma. This invention provides drugs for the management or prevention of bronchial asthma.

### Generating and Manipulating Nucleic Acids and Polypeptides

The invention provides for use of ITK-inhibitory nucleic acids and polypeptides, and nucleic acids encoding them, used alone or in conjunction with the ITK inhibitory proteins of this invention. The invention can be practiced in conjunction with any method or protocol or device known in the art, which are well described in the scientific and patent literature.

The invention provides “nucleic acids” or “nucleic acid sequences” to ITK, including RNAi such as siRNA or miRNA, oligonucleotides, nucleotides, polynucleotides, or any fragments of these, including DNA or RNA (e.g., mRNA, rRNA, tRNA) of genomic or synthetic origin, which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., iRNPs). The invention provides for use of ITK-inhibitory nucleic acids, i.e., oligonucleotides, containing known analogues of natural nucleotides, naturally occurring nucleic acids, synthetic nucleic acids, and recombinant nucleic acids. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. The invention provides for use of ITK-inhibitory deoxyribonucleotide (DNA) or ribonucleotide (RNA) in either single- or double-stranded form. The invention provides for use of nucleic acids containing known analogues of natural nucleotides. The invention provides for use of ITK-inhibitory mixed oligonucleotides comprising an RNA portion bearing 2′-O-alkyl substituents conjugated to a DNA portion via a phosphodiester linkage, see, e.g., U.S. Pat. No. 5,013,830. The invention provides for use of nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene (methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). The invention provides for use of PNAs containing non-ionic backbones, such as N-(2-aminoethyl)glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). The invention provides for use of ITK-inhibitory nucleic acids including genes, polynucleotides, DNA, RNA, cDNA, mRNA, oligonucleotide primers, probes and amplification products.

The invention provides for use of ITK-inhibitory “amino acids” or “amino acid sequences” including an oligopeptide, peptide, polypeptide, or protein sequence, or to a fragment, portion, or subunit of any of these, and to naturally occurring or synthetic molecules. The invention provides for use of ITK-inhibitory “polypeptides” and “proteins” including amino acids joined to each other by peptide bonds or modified peptide bonds, i.e., peptide isosteres, and may contain modified amino acids other than the 20 gene-encoded amino acids. The invention provides for use of ITK-inhibitory “polypeptides” including peptides and polypeptide fragments, motifs and the like. The term also includes glycosylated polypeptides. The invention provides for use of ITK-inhibitory peptides and polypeptides including all “mimetic” and “peptidomimetic” forms.

The nucleic acids used to practice this invention, whether RNA, cDNA, genomic DNA, vectors, viruses or hybrids thereof, may be isolated from a variety of sources, genetically engineered, amplified, and/or expressed/generated recombinantly (recombinant polypeptides can be modified or immobilized to arrays in accordance with the invention). Any recombinant expression system can be used, including bacterial, mammalian, yeast, insect or plant cell expression systems.

As used herein, the term “recombinant” can include nucleic acids adjacent to a “backbone” nucleic acid to which it is not adjacent in its natural environment. “Synthetic” polypeptides or protein are those prepared by chemical synthesis, as described in further detail, below.

Alternatively, nucleic acids can be synthesized in vitro by well-known chemical synthesis techniques, as described in, e.g., Carruthers (1982) Cold Spring Harbor Symp. Quant. Biol. 47:411-418; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Double stranded DNA fragments may then be obtained either by synthesizing the complementary strand and annealing the strands together under appropriate conditions, or by adding the complementary strand using DNA polymerase with a primer sequence.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

The nucleic acids used to practice this invention, whether RNA, iRNA, siRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Alternatively, nucleic acids can be obtained from commercial sources.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., Molecular Cloning: A Laboratory Manual (2nd ed.), Vols. 1-3, Cold Spring Harbor Laboratory, (1989); Current Protocols in Molecular Biology, Ausubel, ed. John Wiley & Sons, Inc., New York (1997); Laboratory Techniques in Biochemistry and Molecular Biology: Hybridization with Nucleic Acid Probes, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In practicing the invention, nucleic acids of the invention or modified nucleic acids of the invention, can be reproduced by amplification. Amplification can also be used to clone or modify the nucleic acids of the invention. Thus, the invention provides amplification primer sequence pairs for amplifying nucleic acids of the invention. One of skill in the art can design amplification primer sequence pairs for any part of or the full length of these sequences.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; and Sooknanan (1995) Biotechnology 13:563-564.

The invention provides for use of ITK-inhibitory peptides and polypeptides isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The invention provides for use of ITK-inhibitory polypeptides that are glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

The invention provides for use of ITK-inhibitory peptides and polypeptides including all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered (e.g., it is ITK-inhibitory).

The invention provides for use of ITK-inhibitory polypeptide mimetic compositions comprising any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

The invention provides for use of ITK-inhibitory polypeptides characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

The invention provides for use of ITK-inhibitory mimetics of acidic amino acids generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl)carbodiimide or 1-ethyl-3(4-azonia-4,4-dimetholpentyl)carbodiimide Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, in one aspect under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl)propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

The invention provides ITK-inhibitory polypeptides as described herein, further altered by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

The invention provides ITK-inhibitory polypeptides or peptides made by solid-phase chemical peptide synthesis methods. For example, assembly of a polypeptides or peptides of the invention can be carried out on a solid support using an Applied Biosystems, Inc. Model 431™ automated peptide synthesizer. Such equipment provides ready access to the polypeptides or peptides of the invention, either by direct synthesis or by synthesis of a series of fragments that can be coupled using other known techniques.

The invention provides ITK-inhibitory proteins lacking a signal peptide, or can lack its endogenous signal peptide and in its place have a heterologous signal peptide.

The invention provides antibodies and methods for using them to inhibit ITK, where “antibody” includes a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; and Yarmush (1992) J. Biochem. Biophys. Methods 25:85-97. Antibodies used to practice this invention include antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody; (v) a dAb fragment (Ward et al., (1989) Nature 341:544-546), which consists of a VH domain; and (vi) an isolated complementarity determining region (CDR). Single chain antibodies are also included by reference in the term “antibody.”

Antisense Oligonucleotides

The invention provides methods for treating asthma and bronchial inflammation using antisense oligonucleotides capable of inhibiting ITK expression and/or activity by targeting mRNA. Strategies for designing antisense oligonucleotides are well described in the scientific and patent literature, and the skilled artisan can design such ITK expression-inhibiting oligonucleotides using the novel reagents of the invention. For example, gene walking/RNA mapping protocols to screen for effective antisense oligonucleotides are well known in the art, see, e.g., Ho (2000) Methods Enzymol. 314:168-183, describing an RNA mapping assay, which is based on standard molecular techniques to provide an easy and reliable method for potent antisense sequence selection. See also Smith (2000) Eur. J. Pharm. Sci. 11:191-198.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl)glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense anti-ITK sequences of the invention (see, e.g., Gold (1995) J. of Biol. Chem. 270:13581-13584).

Inhibitory Ribozymes

The invention provides ribozymes capable of binding ITK message for treating asthma and bronchial inflammation. These ribozymes can inhibit ITK activity by, e.g., targeting mRNA. Strategies for designing ribozymes and selecting the xylanase- and/or glucanase-specific antisense sequence for targeting are well described in the scientific and patent literature, and the skilled artisan can design such ribozymes using the novel reagents of the invention. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The ribozyme of the invention, e.g., an enzymatic ribozyme RNA molecule, can be formed in a hammerhead motif, a hairpin motif, as a hepatitis delta virus motif, a group I intron motif and/or an RNaseP-like RNA in association with an RNA guide sequence. Examples of hammerhead motifs are described by, e.g., Rossi (1992) Aids Research and Human Retroviruses 8:183; hairpin motifs by Hampel (1989) Biochemistry 28:4929, and Hampel (1990) Nuc. Acids Res. 18:299; the hepatitis delta virus motif by Perrotta (1992) Biochemistry 31:16; the RNaseP motif by Guerrier-Takada (1983) Cell 35:849; and the group I intron by Cech U.S. Pat. No. 4,987,071. The recitation of these specific motifs is not intended to be limiting. Those skilled in the art will recognize that a ribozyme of the invention, e.g., an enzymatic RNA molecule of this invention, can have a specific substrate binding site complementary to one or more of the target gene RNA regions. A ribozyme of the invention can have a nucleotide sequence within or surrounding that substrate binding site which imparts an RNA cleaving activity to the molecule.

RNA Interference (RNAi)

In one aspect, the invention provides methods for treating asthma and bronchial inflammation using an RNA inhibitory molecule, a so-called “RNAi” molecule, comprising an ITK sequence. The RNAi molecule can comprise a double-stranded RNA (dsRNA) molecule, e.g., siRNA, miRNA (microRNA) and/or short hairpin RNA (shRNA) molecules. The RNAi molecule, e.g., siRNA (small inhibitory RNA) can inhibit expression of an ITK gene, and/or miRNA (micro RNA) to inhibit translation of ITK message. In one aspect, the RNAi molecule, e.g., siRNA and/or miRNA, is about 11, 12, 13, 14, 15, 16, 17, 18, 19, 20, 21, 22, 23, 24, 25, 26, 27, 28, 29 or more duplex nucleotides in length. While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, see, e.g., Shuey (2002) Drug Discov. Today 7:1040-1046. In one aspect, the invention provides methods to selectively degrade RNA using the RNAi's molecules, e.g., siRNA and/or miRNA, of the invention. The process may be practiced in vitro, ex vivo or in vivo. In one aspect, the RNAi molecules of the invention can be used to generate a loss-of-function mutation in a cell, an organ or an animal.

In one aspect, intracellular introduction of the RNAi is by internalization of a target cell specific ligand bonded to an RNA binding protein comprising an RNAi (e.g., siRNA or microRNA) is adsorbed. The ligand is specific to a unique target cell surface antigen. The ligand can be spontaneously internalized after binding to the cell surface antigen. If the unique cell surface antigen is not naturally internalized after binding to its ligand, internalization can be promoted by the incorporation of an arginine-rich peptide, or other membrane permeable peptide, into the structure of the ligand or RNA binding protein or attachment of such a peptide to the ligand or RNA binding protein. See, e.g., U.S. Patent App. Pub. Nos. 20060030003; 20060025361; 20060019286; 20060019258. In one aspect, the invention provides lipid-based formulations for delivering, e.g., introducing nucleic acids of the invention as nucleic acid-lipid particles comprising an RNAi molecule to a cell, see .g., U.S. Patent App. Pub. No. 20060008910.

Methods for making and using RNAi molecules, e.g., siRNA and/or miRNA, for selectively degrade RNA are well known in the art, see, e.g., U.S. Pat. No. 6,506,559; U.S. Pat. No. 6,511,824; U.S. Pat. No. 6,515,109; U.S. Pat. No. 6,489,127.

### Pharmaceutical Compositions

The invention provides pharmaceutical compositions comprising an ITK-inhibitory nucleic acid or peptide or polypeptide of the invention and a pharmaceutically acceptable excipient. The invention provides for uses of an ITK-inhibitory nucleic acid or peptide or polypeptide of the invention to make a pharmaceutical composition. The invention provides parenteral formulations comprising an ITK-inhibitory nucleic acid or polypeptide of the invention. The invention provides enteral formulations comprising an ITK-inhibitory nucleic acid or polypeptide of the invention. The invention provides methods for treating asthma comprising providing a pharmaceutical composition comprising an ITK-inhibitory nucleic acid or polypeptide of the invention; and administering an effective amount of the pharmaceutical composition to a subject in need thereof.

The pharmaceutical compositions used in the methods of the invention can be administered by any means known in the art, e.g., parenterally, topically, orally, or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Pharmaceutical formulations of the invention can be prepared according to any method known to the art for the manufacture of pharmaceuticals. Such drugs can contain sweetening agents, flavoring agents, coloring agents and preserving agents. A formulation of the invention can be admixtured with nontoxic pharmaceutically acceptable excipients which are suitable for manufacture. Formulations of the invention may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc, or formulated for inhalers, nebulizers, which are devices used to administer medication to people in forms of a liquid mist to the airways, or atomizers. A vaporized medicine can be inhaled through a tube-like mouthpiece, e.g., an inhaler, nebulizer or atomizer; this can have a benefit of allowing surrounding air to mix with the formulation, decreasing the unpleasantness of the vapor, if any.

For example, in one embodiment compositions of the invention can be delivered using a device comprising a nasal actuator with a asymmetric orifice opening that produces bimodal particle size distribution, e.g., delivered using a formulation in the form of a powder packaged under pressure which is released upon activation of an appropriate valve system; as described e.g., in U.S. Pat App Pub No. 20080029084. The compositions of the invention can be formulated as particles in a nebulized solution or powder that lodge along an upper and/or lower or deep respiratory tract. The compositions of the invention can be formulated as dry powders made by spray drying, e.g., with dual nozzles, or spray freeze drying with dual nozzles, or e.g., using a partially friable spray freeze dried powder with a dual particle size distribution, or e.g., by blending of milled freeze-dried or milled powders of two different particle sizes; see e.g., U.S. Pat App Pub No. 20080029084.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a chimeric polypeptide or peptidomimetic of the invention) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration of hydrophobic active agents of the invention. Oil-based suspensions can be formulated by suspending an active agent (e.g., a chimeric composition of the invention) in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In the methods of the invention, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In the methods of the invention, the pharmaceutical compounds can be delivered by transdermally, by a topical route, formulated as applicator sticks, solutions, suspensions, emulsions, gels, creams, ointments, pastes, jellies, paints, powders, and aerosols.

In the methods of the invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In the methods of the invention, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations of the invention can be lyophilized. The invention provides a stable lyophilized formulation comprising a composition of the invention, which can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

Liposomes

The compositions and formulations of the invention can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. For example, in one embodiment, compositions and formulations of the invention are delivered by the use of liposomes having rigid lipids having head groups and hydrophobic tails, e.g., as using a polyethyleneglycol-linked lipid having a side chain matching at least a portion the lipid, as described e.g., in US Pat App Pub No. 20080089928. In another embodiment, compositions and formulations of the invention are delivered by the use of amphoteric liposomes comprising a mixture of lipids, e.g., a mixture comprising a cationic amphiphile, an anionic amphiphile and/or neutral amphiphiles, as described e.g., in US Pat App Pub No. 20080088046, or 20080031937. In another embodiment, compositions and formulations of the invention are delivered by the use of liposomes comprising a polyalkylene glycol moiety bonded through a thioether group and an antibody also bonded through a thioether group to the liposome, as described e.g., in US Pat App Pub No. 20080014255. In another embodiment, compositions and formulations of the invention are delivered by the use of liposomes comprising glycerides, glycerophospholipides, glycerophosphinolipids, glycerophosphonolipids, sulfolipids, sphingolipids, phospholipids, isoprenolides, steroids, stearines, sterols and/or carbohydrate containing lipids, as described e.g., in US Pat App Pub No. 20070148220.

Therapeutically Effective Amount and Dose

The formulations of the invention can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). In the methods of the invention, a pharmaceutical composition is administered in an amount sufficient to treat (e.g., ameliorate) or prevent asthma. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on the dosage and frequency as required and tolerated by the patient. The formulations should provide a sufficient quantity of active agent to effectively treat the treat (e.g., ameliorate) or prevent asthma and/or its symptoms. For example, an exemplary pharmaceutical formulation for oral administration of an ITK-inhibitory nucleic acid or polypeptide of the invention is in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The compositions and formulations of the invention can further comprise other drugs or pharmaceuticals, e.g., compositions for treating asthma and related symptoms or conditions. The methods of the invention can further comprise co-administration with other drugs or pharmaceuticals, e.g., compositions for treating asthma and related symptoms or conditions. For example, the methods and/or compositions and formulations of the invention can be co-formulated with and/or co-administered with antibiotics (e.g., antibacterial or bacteriostatic peptides or proteins), e.g., those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### Kits

The invention provides kits comprising an ITK-inhibitory nucleic acid or polypeptide of the invention, e.g. the pharmaceutical compositions of the invention, including instructions on practicing the methods of the invention, e.g., directions as to indications, dosages, patient populations, routes and methods of administration.

The invention will be further described with reference to the following examples; however, it is to be understood that the invention is not limited to such examples.

### EXAMPLES

### Example 1

### Demonstrating the Efficacy of Compositions of the Invention

The following example describes making and using exemplary ITK-inhibitory compositions of the invention, and provides data demonstrating the efficacy of the methods and compositions of the invention for inhibiting ITK activation, thus treating asthma, asthmatic attacks, respiratory allergic reactions or other pathologic responses, e.g., as induced by an allergens, irritants, poisons or toxins. While the invention is not limited by any particular mechanism of action, the invention provides methods and compositions of the invention for inhibiting ITK activation.

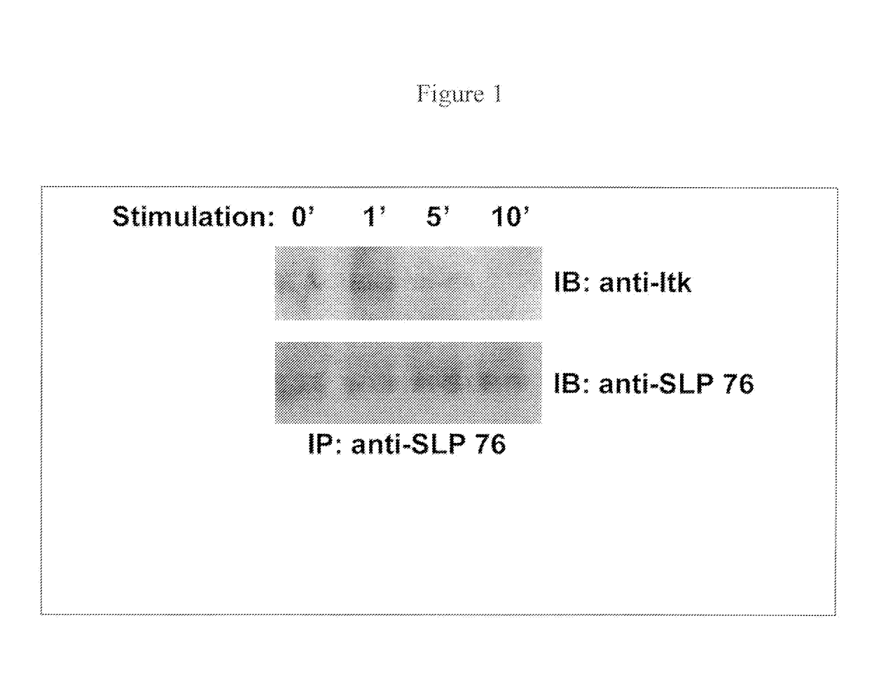

Activation of ITK is initiated by the engagement of antigen by the T cells receptor (TCR) and the activation of the kinase LCK, which trans-phosphorylates ITK at the C-terminus proximal Tyr 511; see e.g., reference (14), below. The activated ITK in turn activates downstream targets that include PLC-gamma 1, the ERK-MAPK pathway, and transcription factors (e.g. T-bet and GATA 3) responsible for the production of type 2 cytokines; see e.g., reference (11), below. One of the requirements for ITK activation is that it associates with an adaptor protein known as SLP76. Thus, upon TCR-mediated activation of T cells, ITK forms an inducible signaling complex with SLP76; see e.g., FIG. 1 and references (15, 16), below.

FIG. 1. Inducible Association between ITK and SLP76: Jurkat T cells were stimulated through their TCR for various periods of time and then lysed. Lysates were treated with antibody to SLP-76 (IP: anti-SLP76) and the immune complexes were resolved by gel electrophoresis. The resolved proteins were transferred onto a membrane where ITK and SLP76 were visualized by probing with specific antibodies reactive with these proteins. First, ITK was probed (IB: anti-ITK) and subsequently SLP76 (IB: anti-SLP76). These data demonstrate that upon T cell stimulation, immunoprecipitation of SLP76 also co-immunoprecipitates ITK. The interpretation is that ITK and SLP76 participate in a stimulation inducible signaling complex. Furthermore, the association of ITK and SLP76 in this complex is time dependent. It happens at one (1) minute post stimulation and then the two proteins dissociate.

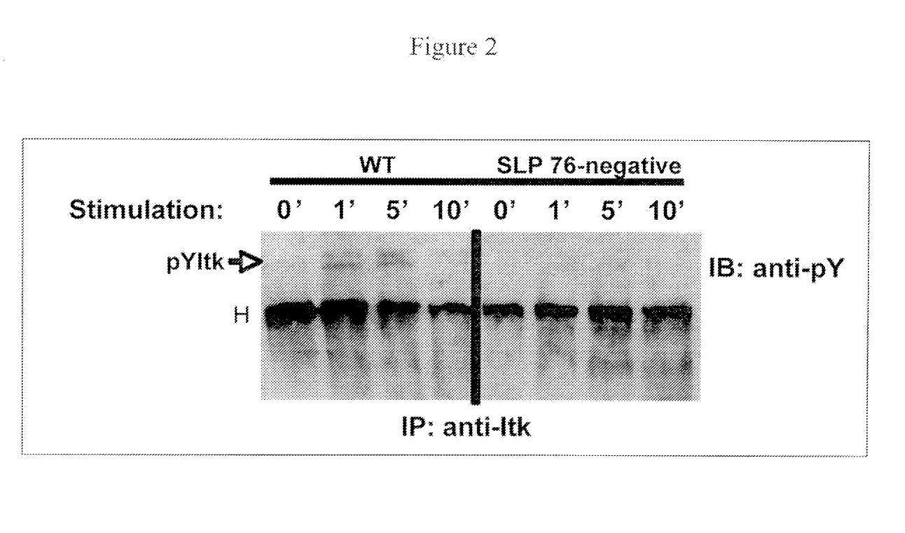

Upon TCR engagement, LCK activates ITK by trans-phosphorylating ITK on Tyr 511, see e.g., reference (14), below. Thus, the phosphorylation status of ITK is an index of its catalytic activity. In view of the inducible association of ITK with SLP76, we asked whether this association is critical for the activation of ITK. To address this question, ITK was immunoprecipitated from either normal Jurkat T cells or mutants lacking expression of SLP76 following their activation. ITK activity was assessed by testing its phosphorylation status. ITK became phosphorylated at 1′ and 5′ post-activation in the normal cells. However, in the SLP76 mutants ITK failed to become phosphorylated, as illustrated in FIG. 2. This is not due to altered kinetics, as we don't see ITK phosphorylation as long as ten minutes following cell stimulation (not shown). These data support the argument that the interaction of ITK with SLP76 is critical for the activation (phosphorylation status) of ITK.

FIG. 2. Activation of ITK depends on the presence of SLP76: Activation of ITK can be assessed by its tyrosine phosphorylation status. The kinase LCK transphosphorylates ITK at the C-terminal tyrosine 511. Jurkat T cells were activated through their TCR for various periods of time as indicated, lysed, and then analyzed by immunoprecipitation as described in FIG. 1. In this experiment ITK was immunoprecipitated with specific anti-ITK antibodies and after resolution by gel electrophoresis, it was probed with anti-phosphotyrosine antibodies (IB: anti-pY).

If activation of T cells induces the formation of a signaling complex that enables the physical association between ITK and SLP76, and if this association is critical for the activation of ITK, a competitive inhibitor of this interaction should be able to abrogate the activation of ITK upon T cell stimulation. The interaction of ITK and SLP76 has been dissected and a specific Proline rich region of SLP76 (amino acids 185-194) involved in this interaction has been identified, see e.g. reference (16), below.

A synthetic peptide encompassing these amino acids has been shown to have a significant affinity for the SH3 domain of ITK, see e.g. reference (17), below. Such synthetic peptide thus constitutes a competitive inhibitor in the interaction between ITK and SLP76. While a synthetic peptide can be translocated into an intracellular environment, even though techniques of peptide translocation have been described (e.g. liposome vehicles), such techniques are not generally very efficient for sufficient delivery and availability of peptide in the intracellular milieu.

Because polyarginine peptide conjugates can be effective vehicles for the intracellular delivery of peptides that inhibit the ITK-SLP76 interaction, thus inhibiting subsequent activation of ITK, the invention provides ITK-inhibitory peptide conjugates, including polyarginine peptide conjugates and equivalents. In one embodiment, the exemplary arginine-SLP76 peptide conjugate: RRRRRRRRRQQPPVPPQRPMA (SEQ ID NO:1) was constructed. The QQPPVPPQRPMA component of the exemplary sequence of this invention represents part of the region of SLP76 that has been shown to interact with the SH3 domain of ITK; see e.g., references (16, 17), below. The reason we selected this part of the SLP76 reactive sequence was because a quantitative measurement of its interaction affinity to ITK's SH3 domain has been determined by chemical shift perturbation assays (Kd=0.77, see e.g., reference (17), below).

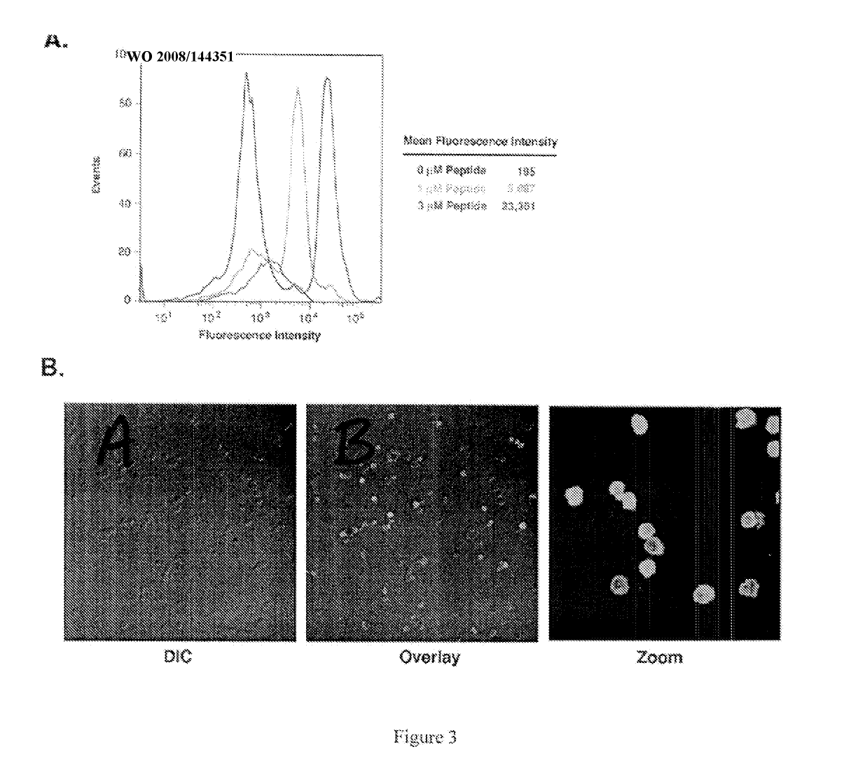

FIG. 3. Intracellular translocation of Arg-SLP76 conjugate peptide: Initially we synthesized this peptide as a fluorescein-conjugate and its effectiveness in entering into Jurkat T cells was tested. Jurkat cells were incubated with 0 μM (buffer control), 1 μM or 3 μM of the exemplary fluoresceinated Arg-SLP76 peptide of the invention, called R9-QQP, for 30 minutes and after thorough washing the cells were analyzed by flow cytometry (FIG. 3A, top panel) or confocal microscopy (bottom panels A, B and C, of FIG. 3B). As noted in FIG. 3A, mean fluorescence intensity was 195, 5,897 and 23,301 for 0 μM (buffer control), 1 μM or 3 μM of the exemplary fluoresceinated peptide, respectively; and the amount of fluorescence intensity is graphically illustrated. In one experiment, at the 1 μM concentration 61.9% of the cells displayed fluorescence in a rather narrow peak, suggesting that the translocation of the peptide was similar among different cells.

To ascertain that the fluorescence was not due to nonspecific peptide sticking on the surface of the cells, we analyzed a sample by laser confocal microscopy. The results shown at the bottom panels of FIG. 3B (bottom panels A, B and C, of FIG. 3B) depict a mid section image of cells that is clearly demonstrating the intracellular location of the fluorescent peptide. Panel A, FIG. 3B displays the differential interference contrast (DIC) image of the field and panel B, FIG. 3B the overlay of the fluorescence and DIC images, and panel C, FIG. 3B is a zoom image.

At higher concentrations of SLP76 peptide of the invention (3, 10, and 30 μM) 100% of the cells displayed positive fluorescence (not shown). Furthermore, at the highest concentration tested the peptide displayed no toxic effects on Jurkat cells as determined by viability measurements. These data indicate an effective uptake of the peptide at the concentrations tested.

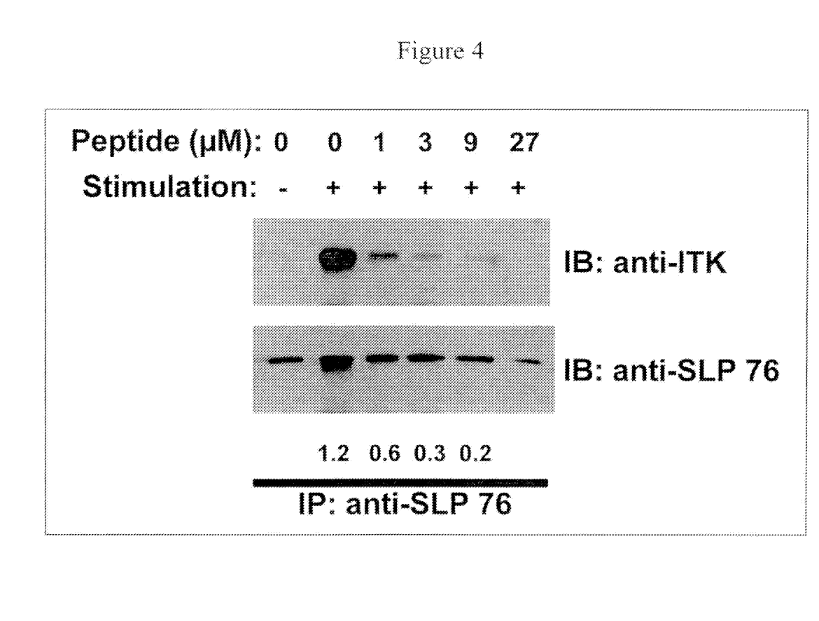

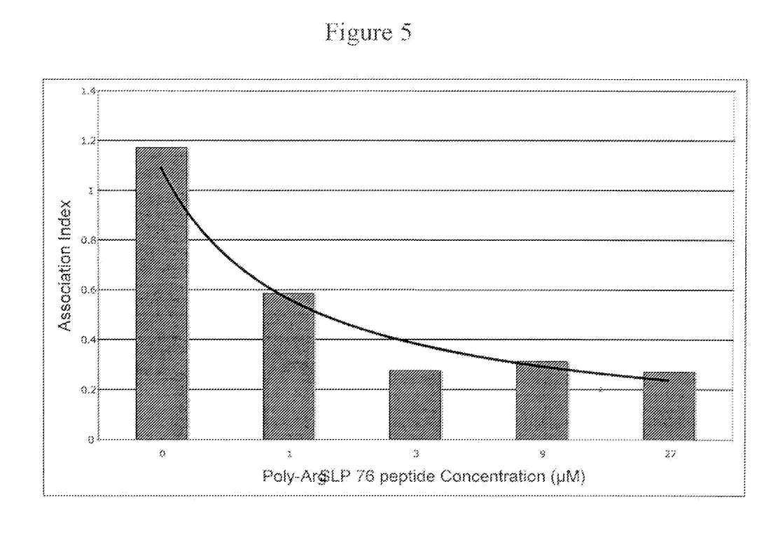

FIG. 4. Inhibition of ITK-SLP76 inducible association by the polyArg-SLP76 peptide conjugate of the invention: We next addressed the important question of whether the presence of the exemplary SLP76 peptide of the invention disrupts the physical association between ITK and SLP76. Jurkat cells were stimulated as described under FIG. 1 above in the presence or absence of various concentrations of the exemplary Arg-conjugated SLP76 peptide of the invention. Stimulation (−, +) was carried out for 1 minute (optimal as per FIG. 1). Following incubation cells were lysed and SLP76 was pulled down with anti-SLP76 specific antibodies (IP: anti-SLP 76). The protein complexes were resolved by gel electrophoresis and then probed with anti-ITK antibodies (IB: anti-ITK) or with anti-SLP 76 antibodies (IB: anti-SLP76). The numbers at the bottom indicate an index of the amount of ITK that co-immunoprecipitated with SLP 76. This index was calculated by correcting the ITK signals for the total of SLP 76 signal in each lane and then calculating the ratio over the non-stimulated control. At 1 μM of peptide there was about 50% inhibition in the ITK-SLP76 interaction. Considering that approximately only 60% of the cells are loaded with peptide at this concentration (see FIG. 3), the peptide causes considerable inhibition in the association between ITK and SLP76. FIG. 5: Summary of inhibition studies of SLP 76-ITK interaction: These observations have been confirmed and a graph depicting the data from the results of several duplicate experiments is graphically illustrated in FIG. 5. Results depict the average of three experiments displayed as Association Index vs. peptide concentration. The association index is defined as in FIG. 4 above; the ratio of the ITK signal over the total SLP 76 signal.

It is important to note that the observed concentration for 50% inhibition in the ITK-SLP 76 (i.e., the ITK-exemplary peptide of the invention) association is about 1 μM, which agrees very well with the calculated Kd (0.77 μM) of the in vitro calculated interaction between this exemplary peptide and the SH3 domain of ITK in chemical shift perturbation assays; see e.g. reference (17), below.

In view of the significant degree of ITK-SLP 76 inhibition mediated by the exemplary arginine-SLP 76 peptide conjugate of the invention, henceforth called R9-QQP, the next question was whether the exemplary R9-QQP peptide can inhibit the phosphorylation of ITK. This is important because it represents an accurate index of ITK activity; see e.g., referenced (14), below. Furthermore, the data described above have been performed by using the Jurkat T cell line. Even though this is a very useful cellular model for research, it is a leukemic cell line and thus, data obtained with this model should be confirmed in a more normal physiological substrate. Therefore, we also tested the effect of R9-QQP on primary mouse splenic lymphocytes.

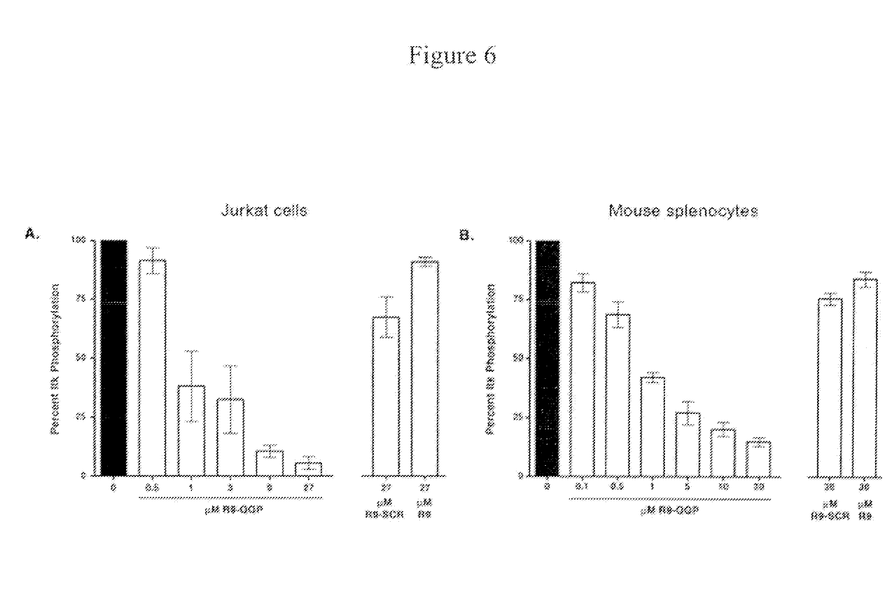

FIG. 6. R9-QQP Specifically Inhibits phosphorylation of ITK on Tyr 511: Jurkat cells or Mouse Splenocytes were incubated with the indicated peptides at the concentrations shown and then stimulated as described above. Following stimulation the cells were stained with specific anti-ITK phosphotyrosine antibodies obtained from a commercial source (BD Biosciences) and following the manufacturer's instructions for intracytoplasmic staining. The stained samples were analyzed in a flow cytometer and the percent phosphorylation related to control (100%) was quantified. The data in FIG. 6 demonstrate that ITK phosphorylation is inhibited by the exemplary R9-QQP in a dose-dependent fashion. Fifty percent inhibition was achieved by approximately 1 μM peptide

An additional concern is the specificity of the R9-QQP effect on ITK phosphorylation. To address this issue we synthesized two control peptides. One is a conjugate of Arginine and the same amino acids as those in the arginine-SLP 76 peptide conjugate, but scrambled in the order of their sequence. This peptide is termed R9-Scrambled arginine-SLP 76 peptide conjugate (R9-SCR). The second control peptide is just the nine Arginine amino acids by themselves. This peptide is termed R9. None of the two control peptides had significant effects on the phosphorylation of ITK in either Jurkat or primary mouse splenic lymphocytes (FIG. 6). This confirms that the effect of R9-QQP on ITK phosphorylation is specific and not due to some unrelated cause of the random order of amino acids.

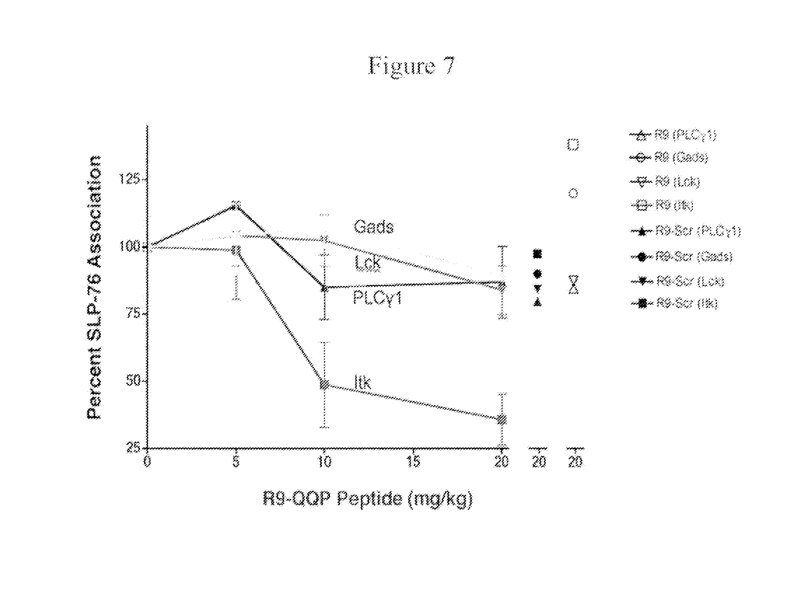

The Arginine-conjugated SLP 76 peptide can be used as a potential drug, and this invention demonstrates that it can be delivered in live animals and be biologically effective. To this end, we injected the peptide into mice and tested its biological effects on the association between ITK and SLP 76, phosphorylation of ITK, and production of cytokines; see Figures, e.g., FIG. 7.

FIG. 7. In vivo delivery of R9-QQP specifically inhibits the inducible ITK-SLP 76 association: Mice were injected intraperitoneally with various concentrations of peptides twenty four hours before sacrifice. A second injection was given the next day, thirty minutes before sacrifice. Splenic cells were isolated and stimulated and the interaction between SLP 76 and the indicated proteins (ITK, PLCγ1, Lck, and Gads) was assessed as described in FIG. 1 above. The data demonstrate that the in vivo delivery of the R9-QQP peptide of this invention disrupted the stimulation-induced association between ITK and SLP 76. Treatment of mice with the two control peptides did not result to any significant inhibition of the association. Importantly, the data in FIG. 7 also demonstrate that the inhibition of ITK and SLP 76 interaction by this peptide of this invention is highly specific for ITK, as other proteins known to interact with SLP 76 by binding to the same domain as ITK binds—but separate parts of the domain—were not affected by the peptide treatment.

In view of the inhibition of ITK-SLP 76 interaction upon in vivo delivery of an exemplary peptide of the invention, the next experiment determined whether the phosphorylation of ITK is also inhibited under this mode of peptide delivery.

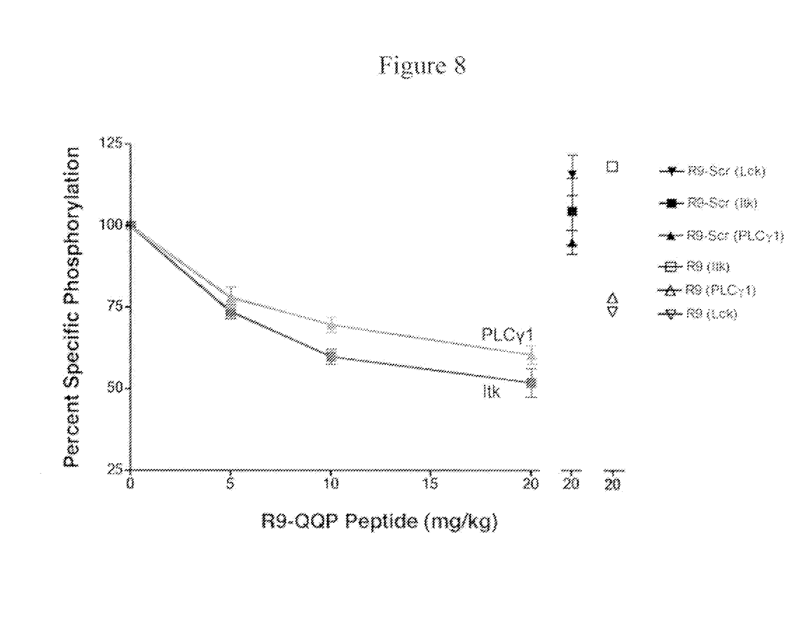

FIG. 8. In vivo delivery of R9-QQP specifically inhibits the inducible phosphorylation of ITK on Tyr 511. Mice were treated with peptide and the isolated splenic lymphocytes activated as described above. Phosphorylation of ITK and PLCγ1 was quantified by use of specific monoclonal antibodies and flow cytometry following the manufacturer's instructions. The data show that ITK phosphorylation is inhibited in a dose-dependent manner by this exemplary peptide of the invention.

Interestingly, the phosphorylation of PLCγ1 is also inhibited but to a slightly lesser extent, see FIG. 8. Phosphorylation of PLCγ1 is a pre-requisite of the activation of this enzyme and it is mediated by the action of ITK after PLCγ1 has associated with SLP 76. Since the exemplary R9-QQP does not inhibit the interaction of PLCγ1 with SLP 76 (FIG. 7), we conclude that the inhibition of PLCγ1 phosphorylation must be due to the inactivation of ITK by the exemplary R9-QQP.

In view of the above data the question is raised whether the exemplary R9-QQP peptide of the invention inhibits the ability of T lymphocytes to produce cytokines. Since ITK is responsible for the regulation of type 2 cytokines (see e.g., reference (24), below) we predicted that the exemplary R9-QQP might inhibit this type of cytokines.

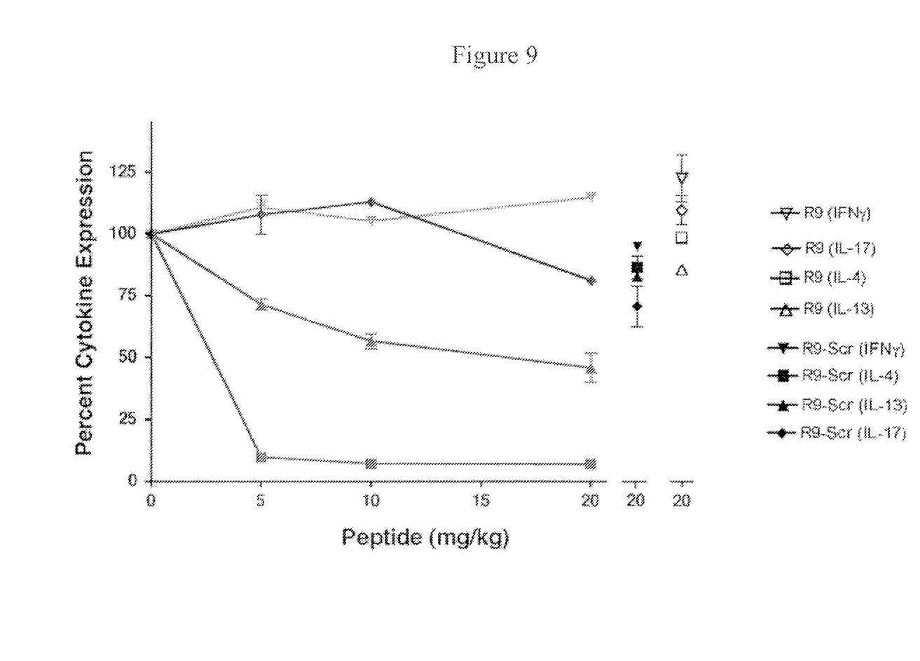

FIG. 9. In vivo delivery of R9-QQP specifically inhibits type 2 cytokines. Mice were treated with peptide and the isolated splenic lymphocytes activated as described above. Intracytoplasmic production of cytokines was quantified by use of specific monoclonal antibodies and flow cytometry following the manufacturer's instructions.

The data indicate that both type 2 cytokines tested (IL-4 and IL-13) were inhibited. The type 1 cytokine IFNγ was not, neither was the newly described cytokine IL-17. Control peptides had no effect. Therefore, the exemplary R9-QQP specifically inhibits type 2 cytokines that are known to be regulated by ITK.

In summary, while the invention is not limited by any particular mechanism of action, these data demonstrate the efficacy of the compositions and methods of this invention, for example: 1) ITK and SLP 76 occur in a signaling complex that is induced upon engagement of the antigen receptor and activation of T cells (this invention's data and ref. (16, 17); 2) The association of ITK with SLP 76 in this signaling complex is absolutely critical for its activation (this invention's data); 3) A region of SLP 76 that binds to the SH3 domain of ITK has been identified (17); 4) this invention provides a synthetic peptide representing this region in conjugate with poly-Arginine (this invention's data); 5) this conjugate peptide enters lymphocytes efficiently (this invention's data); 6) The conjugate peptide inhibits the critical ITK-SLP 76 interaction (this invention's data). 7) The conjugate peptide inhibits the phosphorylation of ITK in both leukemic Jurkat T cells and in primary T cells isolated from the spleens of mice (this invention's data). 8) The peptide can be delivered in vivo and splenic lymphocytes isolated from such treated mice display inhibition of activation-induced ITK-SLP 76 association (this invention's data). 9) The peptide can be delivered in vivo and splenic lymphocytes isolated from such treated mice display inhibition of activation-induced ITK and PLCγ1 phosphorylation (this invention's data). 10) The peptide can be delivered in vivo and splenic lymphocytes isolated from such treated mice display inhibition of type 2 cytokines, but not type 1 cytokines (this invention's data).

In summary, exemplary peptides of the invention were used to inhibit the interaction of ITK and SLP76, and that this interaction is critical for the activation of ITK; and demonstrating that compositions of this invention are effective for inhibiting the activity of ITK. In another aspect, the poly Arg-SLP76 peptide of the invention also inhibits the activity of PLC-gamma-1, which has been shown to be the major target of ITK (see e.g. reference (24), below); ITK has been shown to specifically phosphorylate Tyr 783 on PLC-gamma-1, which is the major tyrosine residue whose phosphorylation regulates PLC-gamma activity (see e.g. reference (25), below).

In another aspect, a peptide encompassing amino acids 224-244 of SLP76 as a poly Arg-SLP76 peptide of the invention inhibits the interaction of SLP76 with another signaling partner, Gads. In this interaction a polyproline rich region of the exemplary SLP76 encompassing amino acids 224-244 interacts with the Gads SH3 domain (26). This interaction is similar to the ITK-SLP76 interaction, but it involves a distinct site of SLP76. Since this region is downstream of the ITK-SLP76 site (amino acids 184-195), the poly Arg-SLP76 peptide of the invention does not affect the association between Gads and SLP76.

The ITK protein inhibitors of the invention also can inhibit downstream events known to be regulated by PLC-gamma-1 in T cells (inhibition of downstream biological effects). The ITK protein inhibitors of the invention also can inhibit targets/downstream biological effects such as intracellular Ca++ mobilization and production of type 2 cytokines (e.g. IL-4 and IL-5) that are known mediators of Eosinophil recruitment in the pathogenesis of bronchial asthma. Flow cytometry can be used for the measurement of Ca++ mobilization and ELISA or intra-cytoplasmic cytokine staining for measuring cytokines.

These data demonstrate that polypeptides of this invention, including the exemplary poly Arg-SLP76 peptide of the invention, can be inhibitory to ITK in vivo. A peptide of the invention can be injected into mice following routine protocols, and then T lymphocytes are isolated from mice to determine the activation of ITK and effects on its downstream targets. Ex vivo activation of T cells from mice treated with the invention's exemplary poly Arg-SLP76 peptide will compromise ITK activation and downstream responses.

In summary: 1) the invention provides for the synthesis of a unique polyArg-SLP76 peptide conjugate, 2) the invention demonstrated the polypeptides of this invention, including the exemplary polyArg-SLP76 peptide conjugate, have efficient penetration and entry into living cells, and 3) the invention demonstrated that polypeptides of this invention can efficiently inhibit the interaction between ITK and SLP76 inside living cells. It should be emphasized that the degree of inhibition we observe in the living cells is in excellent agreement with the reported strength (Kd) of the in vitro ITK-SLP76 interaction thus, making it more biologically relevant.

### CITED REFERENCES

1. Weiss, K. B., A. S. Buist, and S. D. Sullivan. 2000. Asthma's impact on society. Lung Biology in Health and Disease. Marcel Dekker, Inc., New York.
2. Braman, S. S. 2006. The global burden of asthma. Chest 130:4 S-12S.
3. Asthma. Editorial. 2000. In Nature Biotech. IT10-IT11.
4. Maddox, L., and D. A. Schwartz. 2002. The pathophysiology of asthma. Annu Rev Med 53:477-498.
5. Redd, S. C. 2002. Asthma in the United States: burden and current theories. Environ Health Perspect 110 Suppl 4:557-560.
6. Cohn, L., J. A. Elias, and G. L. Chupp. 2004. Asthma: mechanisms of disease persistence and progression. Annu. Rev. Immunol. 22:789-815.
7. Kosaka, Y., M. Felices, and L. J. Berg. 2006. Itk and Th2 responses: action but no reaction. Trends Immunol. 27:453-460.
8. Boulet, L. P., T. R. Bai, A. Becker, D. Berube, R. Beveridge, D. M. Bowie, K. R. Chapman, J. Cote, D. Cockcroft, F. M. Ducharme, P. Ernst, J. M. FitzGerald, T. Kovesi, R. V. Hodder, P. O'Byrne, B. Rowe, M. R. Sears, F. E. Simons, and S. Spier. 2001. What is new since the last (1999) Canadian Asthma Consensus Guidelines? Can Respir J 8 Suppl A:5A-27A.
9. Ernst, P., A. McIvor, F. M. Ducharme, L. P. Boulet, M. FitzGerald, K. R. Chapman, and T. Bai. 2006. Safety and effectiveness of long-acting inhaled beta-agonist bronchodilators when taken with inhaled corticosteroids. Ann. Intern. Med. 145:692-694.
10. Lemiere, C., T. Bai, M. Balter, C. Bayliff, A. Becker, L. P. Boulet, D. Bowie, A. Cartier, A. Cave, K. Chapman, R. Cowie, S. Coyle, D. Cockcroft, F. M. Ducharme, P. Ernst, S. Finlayson, J. M. FitzGerald, F. E. Hargreave, D. Hogg, A. Kaplan, H. Kim, C. Kelm, P. O'Byrne, M. Sears, and A. W. Markham 2004. Adult Asthma Consensus Guidelines Update 2003. Can Respir J 11:9A-18A.
11. Berg, L. J., L. D. Finkelstein, J. A. Lucas, and P. L. Schwartzberg. 2005. Tec family kinases in T lymphocyte development and function. Annu. Rev. Immunol. 23:549-600.
12. Mueller, C., and A. August. 2003. Attenuation of Immunological Symptoms of Allergic Asthma in Mice Lacking the Tyrosine Kinase ITK. J. Immunol. 170:5056-5063.
13. Fowell, D. J., K. Shinkai, X. C. Liao, A. M. Beebe, R. L. Coffman, D. R. Littman, and R. M. Locksley. 1999. Impaired NFATc translocation and failure of Th2 development in Itk-deficient CD4+ T cells. Immunity 11:399-409.
14. Heyeck, S. D., H. M. Wilcox, S. C. Bunnell, and L. J. Berg. 1997. Lck phosphorylates the activation loop tyrosine of the Itk kinase domain and activates Itk kinase activity. J. Biol. Chem. 272:25401-25408.
15. Su, Y. W., Y. Zhang, J. Schweikert, G. A. Koretzky, M. Reth, and J. Wienands 1999. Interaction of SLP adaptors with the SH2 domain of Tec family kinases. Eur. J. Immunol. 29:3702-3711.
16. Bunnell, S. C., M. Diehn, M. B. Yaffe, P. R. Findell, L. C. Cantley, and L. J. Berg. 2000. Biochemical interactions integrating Itk with the T cell receptor-initiated signaling cascade. J. Biol. Chem. 275:2219-2230.
17. Brazin, K. N., D. B. Fulton, and A. H. Andreotti. 2000. A specific intermolecular association between the regulatory domains of a Tec family kinase. J. Mol. Biol. 302:607-623.
18. Green, M., and P. M. Loewenstein. 1988. Autonomous functional domains of chemically synthesized human immunodeficiency virus tat trans-activator protein. Cell 55:1179-1188.
19. El-Andaloussis, S., T. Holm, and U. Langel. 2005. Cell-penetrating peptides: mechanisms and applications. Curr Pharm Des 11:3597-3611.
20. Vives, E., P. Brodin, and B. Lebleu. 1997. A truncated HIV-1 Tat protein basic domain rapidly translocates through the plasma membrane and accumulates in the cell nucleus. J. Biol. Chem. 272:16010-16017.
21. Wender, P. A., D. J. Mitchell, K. Pattabiraman, E. T. Pelkey, L. Steinman, and J. B. Rothbard. 2000. The design, synthesis, and evaluation of molecules that enable or enhance cellular uptake: peptoid molecular transporters. Proc. Natl. Acad. Sci. USA 97:13003-13008.
22. Rothbard, J. B., S. Garlington, Q. Lin, T. Kirschberg, E. Kreider, P. L. McGrane, P. A. Wender, and P. A. Khavari. 2000. Conjugation of arginine oligomers to cyclosporin A facilitates topical delivery and inhibition of inflammation. Nat. Med. 6:1253-1257.
23. Noguchi, H., M. Matsushita, T. Okitsu, A. Moriwaki, K. Tomizawa, S. Kang, S. T. Li, N. Kobayashi, S. Matsumoto, K. Tanaka, N. Tanaka, and H. Matsui. 2004. A new cell-permeable peptide allows successful allogeneic islet transplantation in mice. Nat. Med. 10:305-309.
24. Lucas, J. A., A. T. Miller, L. O. Atherly, and L. J. Berg. 2003. The role of Tec family kinases in T cell development and function. Immunol. Rev. 191:119-138.
25. Kim, H. K., J. W. Kim, A. Zilberstein, B. Margolis, J. G. Kim, J. Schlessinger, and S. G. Rhee. 1991. PDGF stimulation of inositol phospholipid hydrolysis requires PLC-gamma 1 phosphorylation on tyrosine residues 783 and 1254. Cell 65:435-441.
26. Liu, Q., D. Berry, P. Nash, T. Pawson, C. J. McGlade, and S. S. Li. 2003. Structural basis for specific binding of the Gads SH3 domain to an RxxK motif-containing SLP-76 peptide: a novel mode of peptide recognition. Mol. Cell. 11:471-481.

### Example 2

### ITK Inhibitory Nucleic Acid Compositions of the Invention

The invention provides nucleic acid compositions for inhibiting ITK activation; and methods and compositions using these ITK-inhibitory nucleic acids of the invention for inhibiting ITK activation, e.g., to treat asthma or any form of bronchial inflammation, e.g., as induced by an allergen, irritant, poison or toxin. While the invention is not limited by any particular mechanism of action, the invention provides methods and compositions of the invention for inhibiting the expression of human ITK message or protein expression.

The ITK antisense inhibitory nucleic acids used to practice this invention include iRNA such as miRNA and siRNA, antisense nucleic acid and/or ribozymes; and these may be isolated from a variety of sources, genetically engineered, amplified, and/or expressed/generated recombinantly. ITK antisense inhibitory nucleic acids can be expressed using any recombinant expression system, including bacterial, mammalian, yeast, insect or plant cell expression systems. In one embodiment, ITK antisense inhibitory nucleic acids such as iRNA such as miRNA and siRNA, antisense nucleic acid and/or ribozymes, are designed from human sequence, e.g., designed from (SEQ ID NO:10):

| (SEQ ID NO: 10) | |
|---|---|
| 1 | tgcattcttt gccccaaaac tctttccttt ggttgtgcta agaggtgatg cccaaggtgc |
| 61 | accacctttc aagaactgga tcatgaacaa ctttatcctc ctggaagaac agctcatcaa |
| 121 | gaaatcccaa caaaagagaa gaacttctcc ctcgaacttt aaagtccgct tctttgtgtt |
| 181 | aaccaaagcc agcctggcat actttgaaga tcgtcatggg aagaagcgca cgctgaaggg |
| 241 | gtccattgag ctctcccgaa tcaaatgtgt tgagattgtg aaaagtgaca tcagcatccc |
| 301 | atgccactat aaatacccgt ttcaggtggt gcatgacaac tacctcctat atgtgtttgc |
| 361 | tccagatcgt gagagccggc agcgctgggt gctggccctt aaagaagaaa cgaggaataa |
| 421 | taacagtttg gtgcctaaat atcatcctaa tttctggatg gatgggaagt ggaggtgctg |
| 481 | ttctcagctg gagaagcttg caacaggctg tgcccaatat gatccaacca agaatgcttc |
| 541 | aaagaagcct cttcctccta ctcctgaaga caacaggcga ccactttggg aacctgaaga |
| 601 | aactgtggtc attgccttat atgactacca aaccaatgat cctcaggaac tcgcactgcg |
| 661 | gcgcaacgaa gagtactgcc tgctggacag ttctgagatt cactggtgga gagtccagga |
| 721 | caggaatggg catgaaggat atgtaccaag cagttatctg gtggaaaaat ctccaaataa |
| 781 | tctggaaacc tatgagtggt acaataagag tatcagccga gacaaagctg aaaaacttct |
| 841 | tttggacaca ggcaaagaag gagccttcat ggtaagggat tccaggactg caggaacata |
| 901 | caccgtgtct gttttcacca aggctgttgt aagtgagaac aatccctgta taaagcatta |
| 961 | tcacatcaag gaaacaaatg acaatcctaa gcgatactat gtggctgaaa agtatgtgtt |
| 1021 | cgattccatc cctcttctca tcaactatca ccaacataat ggaggaggcc tggtgactcg |
| 1081 | actccggtat ccagtttgtt ttgggaggca gaaagcccca gttacagcag ggctgagata |
| 1141 | cgggaaatgg gtgatcgacc cctcagagct cacttttgtg caagagattg gcagtgggca |
| 1201 | atttgggttg gtgcatctgg gctactggct caacaaggac aaggtggcta tcaaaaccat |
| 1261 | tcgggaaggg gctatgtcag aagaggactt catagaggag gctgaagtaa tgatgaaact |
| 1321 | ctctcatccc aaactggtgc agctgtatgg ggtgtgcctg gagcaggccc ccatctgcct |
| 1381 | ggtgtttgag ttcatggagc acggctgcct gtcagattat ctacgcaccc agcggggact |
| 1441 | ttttgctgca gagaccctgc tgggcatgtg tctggatgtg tgtgagggca tggcctacct |
| 1501 | ggaagaggca tgtgtcatcc acagagactt ggctgccaga aattgtttgg tgggagaaaa |
| 1561 | ccaagtcatc aaggtgtctg actttgggat gacaaggttc gttctggatg atcagtacac |
| 1621 | cagttccaca ggcaccaaat tcccggtgaa gtgggcatcc ccagaggttt tctctttcag |
| 1681 | tcgctatagc agcaagtccg atgtgtggtc atttggtgtg ctgatgtggg aagttttcag |
| 1741 | tgaaggcaaa atcccgtatg aaaaccgaag caactcagag gtggtggaag acatcagtac |
| 1801 | cggatttcgg ttgtacaagc cccggctggc ctccacacac gtctaccaga ttatgaatca |
| 1861 | ctgctggaaa gagagaccag aagatcggcc agccttctcc agactgctgc gtcaactggc |
| 1921 | tgaaattgca gaatcaggac tttagtagag actgagtacc aggccacggg ctgcagatcc |
| 1981 | tgaatggagg aaggatatgt cctcattcca tagagcatta gaagctgcca ccagcccagg |
| 2041 | accctccaga ggcagcctgg cctgtggcat cagtccctga gtcaccatgg aagcagcatc |
| 2101 | ctgaccacag ctggcagtca agccacagct ggagggtcag ccaccaagct gggagctgag |
| 2161 | ccagaacagg agtgatgtct ctgcccttcc tctagcctct tgtcacatgt ggtgcacaaa |
| 2221 | cctcaacctg acagctttca gacagcattc ttgcacttct tagcaacaga gagagacatg |
| 2281 | agtaagaccc agattgctat ttttattgtt atttttaaca tgaatctaaa gtttatggtt |
| 2341 | ccagggactt tttatttgac ccaacaacac agtatcccag gatatggagg caaggggaac |
| 2401 | aaagagcatg agtctttttc caagaaaact ggtgagttaa gtaagattag agtgagtgtg |
| 2461 | ctctgttgct gtgatgctgt cagccacagc ttcctgccgt agagaatgat agagcagctg |
| 2521 | ctcacacagg aggccggata ttctgagaag cagctttatg aggttttaca gagtatgctg |
| 2581 | ctacctctct ccttgaaggg agcatggcga gacccattgg atggattggg gtgaacagtt |
| 2641 | caggtcccat gcttggagca ttgggtatct gatgtctgca ccagaacaag agaacctctg |
| 2701 | acggtggaga accatgtggt gcaagaagag atcttaggtc tcttctttta taccaagctc |
| 2761 | atcttttata ccaagctgtg caggtgacta tgcctcctct tctgcacaga atgcttccac |
| 2821 | cagcatcctg agaagaaatg attacttctg aaaaacatcc ttttttccag cctctgggaa |
| 2881 | tcagcccccc ctctctgcac tatccgatcc tcatcaacag agggcagcat tgtgttggtc |
| 2941 | aatgttccct tggcgagcaa ttgaaacttg tttaggccct agggttgagc aattttaagg |
| 3001 | ttgagactcc aagtctccta aaattctagg agagaaataa agagtctgtt tttgctcaaa |
| 3061 | ccatcaggat ggaaacagtc aggcactgac tggggtgctt ccaagaggca tgagagtgcc |
| 3121 | tactctggct tgagcacttc tatatgcaag gtgaatatgt actgagctag gagacttccc |
| 3181 | tgcaaaatct ctgttcaccc tgggttcaca tccccatgag gtaatattat tattcccatt |
| 3241 | ttacaaataa tgtaactgag gctttaaaaa gccaagacat ctgcccaaag tgatggaact |
| 3301 | agaaagtcta gagctggtat tctagcccaa atctgtctga ccgcaataca cagattcttt |
| 3361 | attcctattc gacactggct tctactgaaa atgaaacgga ttgcagaggg aataaataca |
| 3421 | aagatggaaa gccagtaaag aagtcagtat agaaccacta gcgaatagtg ttgctctggc |
| 3481 | acagaccact gtggttgatg gcatggccct ccaacttgga ataggatttt ccttttccta |
| 3541 | ttctgtatcc ttaccttggt catgttaatg actttggagt tattcagtta atgacccttt |
| 3601 | aattctcaca accaaccagt catgttgctt gaagccattt atagacgagc ttcaaagcaa |
| 3661 | ctttaaaaga ttcttctgta gaagtatgag ttcttccttt aattatcatt ccaactttca |
| 3721 | gctgtagtct tcttgaacac ttcatgagga gggacattcc ctgatataag agaggatggt |
| 3781 | gttgcaattg gctctttcta aatcatgtga cgttttgact ggcttgagat tcagatgcat |
| 3841 | aatttttaat tataattatt gtgaagtgga gagcctcaag ataaaactct gtcattcaga |
| 3901 | agatgatttt actcagctta tccaaaatta tctctgttta ctttttagaa ttttgtacat |
| 3961 | tatcttttgg gatccttaat tagagatgat ttctggaaca ttcagtctag aaagaaaaca |
| 4021 | ttggaattga ctgatctctg tggtttggtt tagaaaattc ccctgtgcat ggtattacct |
| 4081 | ttttcaagct cagattcatc taatcctcaa ctgtacatgt gtacattctt cacctcctgg |
| 4141 | tgccctatcc cgcaaaatgg gcttcctgcc tggtttttct cttctcacat tttttaaatg |
| 4201 | gtcccctgtg tttgtagaga actcccttat acagagtttt ggttctagtt ttatttcgta |
| 4261 | gattttgcat tttgtacctt ttgagactat gtatttatat ttggatcaga tgcatattta |
| 4321 | ttaatgtaca gtcactgcta gtgttcaaaa taaaaatgtt acaaat |

In one embodiment, ITK antisense inhibitory nucleic acids such as iRNA such as miRNA and siRNA, antisense nucleic acid and/or ribozymes, are designed to inhibit the expression of human ITK, e.g., designed as nucleic acids antisense to nucleic acid sequence encoding human ITK, e.g., as set forth in SEQ ID NO:11 (see, e.g., Tanaka (1993) FEBS Lett. 324 (1):1-5; Genbank accession code NM_{—}005546):

(SEQ ID NO: 11)
MNNFILLEEQLIKKSQQKRRTSPSNFKVRFFVLTKASLAYFEDRHGKKR
TLKGSIELSRIKCVEIVKSDISIPCHYKYPFQVVHDNYLLYVFAPDRES
RQRWVLALKEETRNNNSLVPKYHPNFWMDGKWRCCSQLEKLATGCAQYD
PTKNASKKPLPPTPEDNRRPLWEPEETVVIALYDYQTNDPQELALRRNE
EYCLLDSSEIHWWRVQDRNGHEGYVPSSYLVEKSPNNLETYEWYNKSIS
RDKAEKLLLDTGKEGAFMVRDSRTAGTYTVSVFTKAVVSENNPCIKHYH
IKETNDNPKRYYVAEKYVFDSIPLLINYHQHNGGGLVTRLRYPVCFGRQ
KAPVTAGLRYGKWVIDPSELTFVQEIGSGQFGLVHLGYWLNKDKVAIKT
IREGAMSEEDFIEEAEVMMKLSHPKLVQLYGVCLEQAPICLVFEFMEHG
CLSDYLRTQRGLFAAETLLGMCLDVCEGMAYLEEACVIHRDLAARNCLV
GENQVIKVSDFGMTRFVLDDQYTSSTGTKFPVKWASPEVFSFSRYSSKS
DVWSFGVLMWEVFSEGKIPYENRSNSEVVEDISTGFRLYKPRLASTHVY
QIMNHCWKERPEDRPAFSRLLRQLAEIAESGL

### Example 3

### Compositions of the Invention with Arginine Peptidomimetics

The invention provides peptide conjugates for inhibiting ITK activation; and methods and compositions using these ITK-inhibitory peptide conjugates of the invention for inhibiting ITK activation, e.g., to treat asthma or any form of bronchial inflammation, e.g., as induced by an allergen, irritant, poison or toxin. In alternative embodiments, the peptide conjugates of the invention comprise arginine residues, or arginine peptidomimetics, e.g., arginine-containing peptidomimetic compounds, or any equivalent a guanidino group-containing basic amino acid.

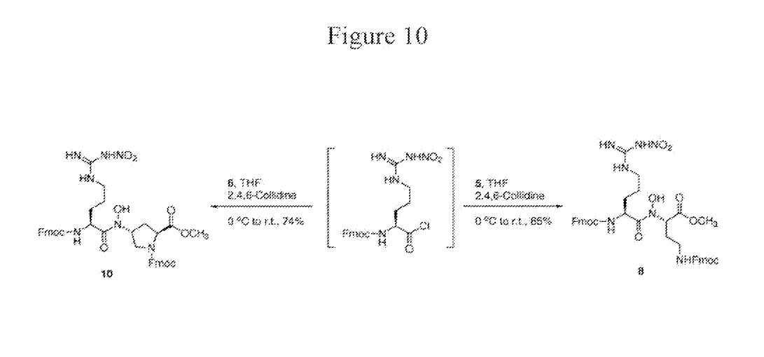

Synthesis of peptidomimetics for use in this invention, including arginine peptidomimetics, e.g., arginine-containing hydroxamates, can be by any peptide coupling methods known in the art. For example, see e.g., Seo (2006) Tetrahedron Letters 47(24):4069-4073, describing a protocol comprising using Fmoc-Arg(NO₂)—Cl prepared at low temperature to undergo intramolecular δ-lactam formation to provide a hydroxamate (compounds 8 and 10 in FIG. 10) in good yields. Fmoc and N-nitro protecting groups can be easily removed. See also, e.g., Lim (1998) Chem. Commun. 2375-2376.

Synthesis of peptidomimetics for use in this invention can comprise use of synthetic methods and compounds comprising use of amino amides, peptides and peptidomimetics, e.g., using amino amide derivatives prepared by a process comprising a one-step, three-component reaction of a glyoxamide, an amine, and an organoboron derivative, as described e.g., in U.S. Pat. No. 7,247,701. Synthesis of peptidomimetics for use in this invention can comprise use of methods for preparing a peptide having a stable, internally constrained alpha-helical, beta-sheet/beta-turn, as described e.g., in U.S. Pat. No. 7,202,332.

A number of embodiments of the invention have been described. Nevertheless, it will be understood that various modifications may be made without departing from the spirit and scope of the invention. Accordingly, other embodiments are within the scope of the following claims.

1. An isolated, synthetic or recombinant polypeptide or peptide comprising or consisting of
(a) an amino acid sequence comprising or consisting of the formula:
R₁-QQPPV (SEQ ID NO: 2)-R₂,
R₁-GlnGlnProProVal (SEQ ID NO: 2)-R₂,
R₁-GlnGlnProProR₃ (SEQ ID NO: 9)-R₂,
R₁-GlnGlnProProR₄ (SEQ ID NO: 9)-R₂,
or
R₁-R₅R₆GlnProProR₄ (SEQ ID NO: 9)-R₂,
wherein in R₁ and R₂, R is independently an arginine amino acid residue, an arginine peptidomimetic residue, a ketopiperazine, amidinophenylalanine residues, a guanidino group-containing basic amino acid, or an arginine amino acid equivalent; and 1 and 2 of R₁ and R₂ is an integer between 1 and 50, or 1 and 2 of R₁ and R₂ are independently selected from 1, 2, 3, 4, 5, 6, 7, 8, 9, 10, 11, 12, 13, 14, 15, 16, 17, 18, 19 or 20 or more arginine amino acid residues, arginine peptidomimetics residues, ketopiperazine residues, amidinophenylalanine residues, guanidino group-containing basic amino acids or arginine amino acid equivalents,
and R₃ is a hydrophobic amino acid residue, or R₄ is valine (val, or V), leucine (Leu, or L), isoleucine (Ile, or I), or alanine (ala, or A), or equivalent;
and R₄ is a nonpolar amino acid residue, or R₄ is glycine (Gly, or G), alanine (ala, or A), valine (val, or V), leucine (Leu, or L), isoleucine (Ile, or I), methionine (met, or M), phenylalanine (Phe, or F), tryptophan (trp, or W) or proline (pro, or P), or equivalent;
and R₅ and R₆ are a polar amino acid residue, or R₅ and R₆ are independently serine (ser, or S), threonine (thr, or T), cysteine (cys, or C), tyrosine (tyr, or Y), asparagine (asp, or N), or glutamine (Gln, or Q);

| | |
|---|---|
| (b) amino acid sequence | |
| RRRRRRRRRQQPPVPPQRPMA, | (SEQ ID NO: 1) |
| QQPPV, | (SEQ ID NO: 2) |
| QQPPVPPQRPM, | (SEQ ID NO: 3) |
| QQPPVPPQRP; | (SEQ ID NO: 4) |
| QQPPVPPQR, | (SEQ ID NO: 5) |
| QQPPVPPQ, | (SEQ ID NO: 6) |
| QQPPVPP, | (SEQ ID NO: 7) |
| or | |
| QQPPVP; | (SEQ ID NO: 8) |

(c) the polypeptide or peptide of (b) further comprising a poly-arginine amino acid residue moiety, or equivalent;
(d) the polypeptide or peptide of (c), wherein the poly-arginine moiety amino acid residues are located amino terminal, carboxy terminal, or amino terminal and carboxy terminal to the polypeptide or peptide;
(e) a polypeptide or peptide conjugate comprising a non-functional subsequence of SEQ ID NO:1, SEQ ID NO:3, SEQ ID NO:4, SEQ ID NO:5, SEQ ID NO:6, SEQ ID NO:7 or SEQ ID NO:8, and comprising the motif QQPPV (SEQ ID NO:2) and a poly-arginine moiety, or equivalent;
(f) a peptidomimetic of the polypeptide or peptide of any of (a) to (d);
(g) the polypeptide or peptide of any of (a) to (e), or peptidomimetic of (f), further comprising, or modified by: acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation and/or arginylation;
(h) the polypeptide or peptide of any of (a) to (e), or peptidomimetic of (f), further comprising a second domain or moiety;
(i) the polypeptide or peptide of (h), wherein the second domain or moiety comprises a targeting agent;
(j) the polypeptide or peptide of (i), wherein the targeting agent comprises an antibody Fc domain or an antibody that binds to an Fc receptor, or a chimeric protein comprising two or more antibody Fc domains; or
(k) the polypeptide or peptide of (h), wherein the second domain or moiety comprises an Fc domain, a protein C, an antibacterial or bacteriostatic peptide or protein, an antibiotic, a cytokine, an immunoregulatory agent, an anti-inflammatory agent, a complement activating agent, a carbohydrate-binding domain or a combination thereof.

2. A chimeric protein comprising
(A) (a) a first domain comprising the isolated, synthetic or recombinant polypeptide or peptide of claim 1, and at least a second domain or moiety; (b) the chimeric protein of (a), wherein the chimeric protein comprises a recombinant fusion protein; or (c) the chimeric protein of (a) or (b), wherein the second domain or moiety comprises a targeting agent.
(B) the chimeric protein of (A), wherein the targeting agent comprises an antibody Fc domain or an antibody that binds to an Fc receptor, or a chimeric protein comprising two or more antibody Fc domains;
(C) the chimeric protein of (A) or (B), wherein the at least second domain or moiety comprises an Fc domain, a protein C, an antibacterial or bacteriostatic peptide or protein, an antibiotic, a cytokine, an immunoregulatory agent, an anti-inflammatory agent, a complement activating agent, a carbohydrate-binding domain or a combination thereof;
(D) the chimeric protein of any of (A) to (C), wherein the chimeric protein comprises a recombinant, peptidomimetic or synthetic protein; or
(E) the chimeric protein of any of (A) to (D), wherein the first domain is joined to the second domain or moiety by a chemical linking agent.

3. A composition comprising (a) the isolated, synthetic or recombinant polypeptide or peptide of claim 1 and a second composition; or (b) the composition of (a), wherein the second composition comprises a liquid, a lipid or a powder.

4. A liposome comprising (a) the isolated, synthetic or recombinant polypeptide or peptide of claim 1; or (b) the liposome of (a), wherein the liposome is formulated with a pharmaceutically acceptable excipient.

5. A pharmaceutical composition comprising: the isolated, synthetic or recombinant polypeptide or peptide of claim 1; and, a pharmaceutically acceptable excipient.

6. An inhalant or spray formulation comprising: the isolated, synthetic or recombinant polypeptide or peptide of claim 1, and, a pharmaceutically acceptable excipient.

7. A parenteral or enteral formulation comprising: the isolated, synthetic or recombinant polypeptide or peptide of claim 1, and, a pharmaceutically acceptable excipient.

8. (canceled)

9. A method for treating, ameliorating or preventing a bronchial inflammation and/or an asthma, or an asthmatic incident, in an individual in need thereof, comprising:
(A) (a) providing the isolated, synthetic or recombinant polypeptide or peptide of claim 1; and
(b) administering an effective amount of (a) to the individual, thereby treating the asthma; or,
(B) the method of (A), wherein the asthma is a human bronchial asthma, and/or the bronchial inflammation is caused by a toxin, poison or poison gas, and/or allergen.

10. A method for suppressing inflammation of bronchial tubes by inhibiting the activation of T-helper cells in an individual, comprising:
(A) (a) providing the isolated, synthetic or recombinant polypeptide or peptide of claim 1; and
(b) administering an effective amount of the composition of (a) to the individual; or
(B) the method of (A), wherein the individual is a human.

11. A method for suppressing or preventing an immune response in an individual in response to inhalation of an allergen by the individual, comprising:
(A) (a) providing the isolated, synthetic or recombinant polypeptide or peptide of claim 1; and
(b) administering an effective amount of the composition of (a) to the individual; or
(B) the method of (A), wherein the individual is a human.

12. A method for suppressing or preventing activation of a CD4⁺ T cell, comprising:
(A) (a) providing the isolated, synthetic or recombinant polypeptide or peptide of claim 1; and
(b) contacting an effective amount of the composition of (a) to the CD4⁺ T cell; or
(B) the method of (A), wherein the CD4⁺ T cell is isolated or in an individual with asthma, bronchial asthma, a bronchial inflammation, or a bronchial inflammation caused by a toxin, poison or poison gas, and/or allergen.

13. A method for suppressing or preventing secretion from an inflammatory cell a prostaglandin, a type-2 cytokines and/or a leukotriene, comprising:
(A) (a) providing the isolated, synthetic or recombinant polypeptide or peptide of claim 1; and
(b) contacting an effective amount of the composition of (a) to the inflammatory cell; or
(B) the method of (A), wherein the inflammatory cell is isolated or in an individual with asthma, bronchial asthma, a bronchial inflammation, or a bronchial inflammation caused by a toxin, poison or poison gas, and/or allergen.

14. An isolated, synthetic or recombinant nucleic acid comprising or consisting of:
(a) a nucleic acid sequence encoding the polypeptide or peptide of claim 1;
(b) the nucleic acid sequence of (a), and further comprising or consisting of nucleic acid sequence encoding a polypeptide antigen, label or tag;
(c) the nucleic acid sequence of (b), wherein the polypeptide antigen, label or tag comprises or consists of a fluorescent or a detectable protein, or an enzyme, or an enzyme that generates a detectable agent or moiety.

15. A host cell, a vector, a cloning or expression vector, an expression cassette, a plasmid, a phage, or a recombinant virus, comprising the isolated or recombinant nucleic acid of claim 14.

16. (canceled)

17. A non-human transgenic animal comprising
(a) a nucleic acid of claim 14; or
(b) the non-human transgenic animal of (a), wherein the animal is a mouse or a rat.

18. (canceled)

19. An inhaler, nebulizer or atomizer comprising the isolated, synthetic or recombinant polypeptide or peptide of claim 1.

20. A pharmaceutical composition comprising
(a) a human ITK antisense inhibitory nucleic acid; and, a pharmaceutically acceptable excipient, wherein the antisense inhibitory nucleic acid is inhibitory to the expression of SEQ ID NO:10, or
(b) the human ITK antisense inhibitory nucleic acid of (a), comprising or consisting of an iRNA, an miRNA, an siRNA, an antisense nucleic acid and/or a ribozyme.

21. An inhalant or spray formulation, or a parenteral or enteral formulation, comprising the pharmaceutical composition of claim 20; and, a pharmaceutically acceptable excipient.

22-23. (canceled)

24. A method for treating, ameliorating or preventing a bronchial inflammation and/or an asthma, or an asthmatic incident, in an individual in need thereof, comprising:
(A) (a) providing the pharmaceutical composition of claim 20; and
(b) administering an effective amount of (a) to the individual, thereby treating the asthma; or,
(B) the method of (A), wherein the asthma is a human bronchial asthma, and/or the bronchial inflammation is caused by a toxin, poison or poison gas, and/or allergen.

25. A method for suppressing inflammation of bronchial tubes by inhibiting the activation of T-helper cells in an individual, comprising:
(A) (a) providing the pharmaceutical composition of claim 20; and
(b) administering an effective amount of the composition of (a) to the individual; or
(B) the method of (A), wherein the individual is a human.

26-33. (canceled)
